(19) United States
(12) Patent Application Publication
(10) Pub. No.: US 2022/0080405 A1
(43) Pub. Date: Mar. 17, 2022

(54) ASSAY DEVICE

(71) Applicant: ABBOTT RAPID DIAGNOSTICS INTERNATIONAL UNLIMITED COMPANY, Dublin (IE)

(72) Inventors: Rookiem LU, Dublin (IE); Hyde Hu, Dublin (IE); Anne Becknell, Dublin (IE); Nicole Haupt Jabri, Dublin (IE); Roger King, Dublin (IE); Linda Verdi, Dublin (IE); Neil Quitoviera, Dublin (IE); Joke Hu, Dublin (IE); Susan Xue, Dublin (IE)

(21) Appl. No.: 17/419,369

(22) PCT Filed: Nov. 26, 2019
(86) PCT No.: PCT/CN2019/120875

§ 371 Date: Jun. 29, 2021

(30) Foreign Application Priority Data
Dec. 31, 2018 (CN) PCT/CN2018/126041

(51) Int. Cl. B01L 3/00; G01N 33/543; G01N 33/94; B33Y 80/00; B29C 45/00

(52) U.S. Cl. CPC B01L 3/5023; B29L 2031/752; G01N 33/94; B33Y 80/00; B29C 45/0053; B01L 2300/025; B01L 2200/026; B01L 2200/0689; B01L 2200/12; B01L 2200/141; B01L 2200/143; B01L 2200/16; B01L 2300/042; B01L 2300/0663; B01L 2300/069; B01L 2300/0825; B01L 2300/0858; B01L 2300/12; B01L 2400/0457; G01N 33/54388

(57) ABSTRACT

Provided herein is technology relating to detecting one or more analytes in a sample and particularly, but not exclusively, to devices, methods, systems, and kits for detecting one or more drugs-of-abuse analytes in a biological sample.

### FIELD

Provided herein is technology relating to detecting one or more analytes in a sample and particularly, but not exclusively, to devices, methods, systems, and kits for detecting one or more drugs-of-abuse analytes in a biological sample.

### BACKGROUND

Sample collection and assay devices for clinical or home use are available. These devices find use in various applications, including the detection of drugs, biological compounds, antibodies, and etiological agents. Many of these devices are complex, vulnerable to tampering, and/or test a limited set of analytes. Also, manufacture of many present devices is expensive. Accordingly, improved technologies are needed.

### SUMMARY

In some embodiments, the technology described herein provides an assay device comprising a chamber configured to collect and/or contain a sample. In some embodiments, the assay device further comprises a reservoir configured to receive a portion of the sample from the chamber. In some embodiments, the assay device further comprises a reservoir seal configured to limit the amount of sample accessible to the reservoir. In some embodiments, the reservoir further comprises a test device for testing the sample for the presence, absence, and/or quantity of an analyte. In some embodiments, the chamber and the reservoir are in fluid communication through a sample passage slot. In some embodiments, the assay device comprises components, features, and materials, and finds use in various applications, as described in U.S. Pat. Nos. 8,865,458; 8,394,626; and 7,560,272, each of which is incorporated herein by reference in its entirety.

In addition to components, features, materials, and uses as described in the patents referenced above, the present technology provides one or more improvements. For example, in some embodiments, the present technology provides an assay device comprising an indication structure configured to produce a vibration (e.g., an audible sound and/or a haptic feedback) when a lid securely seals the chamber, e.g., as described in U.S. Pat. No. 9,730,646, which is incorporated herein by reference in its entirety.

In some embodiments, the present technology provides an assay device configured to test for an increased number of drugs of abuse in a sample than are tested by previous devices. For example, in some embodiments the present technology provides an assay device that simultaneously tests for the presence, absence, and/or quantity of 14 or more drugs of abuse (e.g., 14, 15, 16, 17, 18, 19, 20, 21, 22, 23, 24, 25, or more drugs of abuse) in a sample. In some embodiments, the present technology provides an assay device comprising 6 or more test strips (e.g., 6, 7, 8, 9, 10, 11, 12, or more test strips). In some particular embodiments, the assay device comprises 6, 7, or 8 test strips. In some embodiments, the assay device comprises 9, 10, 11, 12, 13, 14, or 15 or more test strips. In some embodiments, the present technology provides an assay device configured to test for specifically relevant combinations of two or more drugs, e.g., heroin and/or a heroin metabolite and fentanyl; and/or oxycodone, propoxyphene, and tramadol in a sample.

In some embodiments, the present technology provides an assay device configured to validate a sample, e.g., to verify that a sample provided and/or obtained by a subject is not adulterated, substituted, diluted, modified, and/or damaged (e.g., by excessive heat, cold, light exposure, chemical exposure, enzyme exposure, etc.), e.g., to verify that the sample is consistent with a normal sample except for the presence of analytes or metabolites thereof. In particular, some embodiments of the technology comprise a specimen validity testing strip configured to test for four aspects of sample validity simultaneously on said single strip, e.g., in some embodiments a test strip assays a sample for: 1) presence of an oxidant (e.g., bleach) in the sample; 2) presence of creatinine in the sample; 3) density (e.g., specific gravity) of the sample; and 4) hydrogen ion concentration (e.g., pH) of the sample.

In some embodiments, the present technology provides an assay device that has an improved resistance to flooding of the reservoir and thus improved resistance to flooding of the test strips. In particular, some embodiments of the technology comprise a sample passage slot that is not rectangular. For instance, in some embodiments, the technology provides an assay device comprising a trapezoidal, triangular, or arched sample passage slot. In some embodiments, the test strips of the assay device are not flooded when the assay device is tilted from vertical (e.g., at an angle of up to approximately 30° (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30° from vertical).

In some embodiments, the technology provides an assay device comprising a vent hole to allow flow of gas (e.g., air) from the reservoir to the chamber as the sample flows through the sample passage slot. In some embodiments, the assay device comprises a sample passage slot that is in the shape of a triangle and a portion of the triangle (e.g., a top portion) allows flow of gas (e.g., air) from the reservoir to the chamber as the sample flows through a portion (e.g., a bottom portion) of the triangular sample passage slot.

In some embodiments, the present technology provides an assay device that does not comprise a membrane (e.g., a raised polyester membrane) on the bottom (“floor”) wall of the reservoir (e.g., the technology provides a “membrane-free” assay device), e.g., as provided in related embodiments, e.g., as described in U.S. Pat. Nos. 8,865,458; 8,394,626; and 7,560,272, each of which is incorporated herein by reference in its entirety. Manufacture and assembly of embodiments of a membrane-free assay device are less expensive and more efficient relative to previous embodiments comprising a membrane (e.g., a raised polyester membrane).

In some embodiments, the size and/or shape of the trapezoidal or arched sample passage slot and the vent hole promote flow of sample from the chamber to the reservoir. Accordingly, the technology provides embodiments of an improved device wherein the size and/or shape of the trapezoidal or arched sample passage slot and the vent hole promote sample flow from the chamber to the reservoir instead of providing a membrane (e.g., a raised polyester membrane) on the floor of the reservoir to draw sample into the reservoir. In some embodiments comprising a triangular sample passage slot, the size and/or shape of the triangular sample passage slot promote flow of sample from the chamber to the reservoir. For example, in some embodiments, the top of the triangular sample passage slot acts as a vent and the bottom of the triangular sample passage slot allows passage of sample.

Furthermore, embodiments of the assay device comprise improved test strips. In some embodiments, the test strips have the same size as other, similar assay devices, thus providing a uniform, standardized test strip for multiple types of assay devices. In particular embodiments, the test strips comprise a sample pad that is more than 19 mm in length (e.g., 20, 21, 22, 23, 24, 25, 26, 27, 28, 29, or 30 mm in length). In particular embodiments, the test strips are assembled from layers to decrease the susceptibility of the test strips to delamination. For example, in some embodiments, the overlap of a first layer (e.g., an adhesive tape) and a second layer (e.g., an immunochromatographic matrix on an impermeable backing layer) is increased to minimize and/or eliminate the delamination of the test strip. In some embodiments, the overlap of a first layer (e.g., an adhesive tape) and a second layer (e.g., an immunochromatographic matrix on an impermeable backing layer) is approximately 2 to 3 mm (e.g., 2.0, 2.1, 2.2, 2.3, 2.4, 2.5, 2.6, 2.7, 2.8, 2.9, or 3.0 mm). In some embodiments, the test strips are thinner than previous test strips, e.g., in some embodiments the test strips are approximately 3.5 mm wide (e.g., 3.3, 3.4, 3.5, 3.6, or 3.7 mm wide).

In some embodiments, the present technology provides an assay device that is more sensitive than previous assay devices, e.g., in some embodiments the present assay device identifies the presence of quantities of drugs in a sample that are lower than the quantities of drugs identified as a positive result by previous devices.

In some embodiments, the present technology provides an assay device that is designed to decrease the costs associated with manufacture of the assay device. For example, embodiments of the assay device comprise a flatter, thinner test strip support panel, which provides an assay device that is more easily assembled by automated methods. In some embodiments, the present technology provides an assay device that is designed to be read by an automated reader. For example, embodiments provide an assay device comprising a uniformly flat surface covering the test strips that promotes uniform contact with an optical reader system. In some embodiments, the assay device is designed so that the test strips are closer to the surface of the assay device so that they are closer to an optical reader system when the flat surface covering the test strips is contacted to the optical reader system.

The technology finds use in a variety of fields and applications, e.g., in forensic use, for employment and insurance, and as an in vitro diagnostic.

Accordingly, in some embodiments the technology provides an assay device comprising a chamber configured for collecting and holding a sample (e.g., wherein the chamber comprises an indication structure; a reservoir (e.g., wherein the reservoir comprises a test device (e.g., wherein the test device comprises 6 or more channels (e.g., 6, 7, 8, 9, 10, 11, 12, 13, 14, 15, or more channels) configured to hold 6 or more test strips (e.g., 6, 7, 8, 9, 10, 11, 12, 13, 14, 15, or more test strips))); and a non-rectangular sample passage slot fluidly connecting said chamber and said reservoir. In some embodiments, the non-rectangular sample passage slot has a trapezoidal shape. In some embodiments, the trapezoidal shape has a first base that is 20-40 mm long (e.g., 20, 21, 22, 23, 24, 25, 26, 27, 28, 29, 30, 31, 32, 33, 34, 35, 36, 37, 38, 39, or 40 mm). In some embodiments, the trapezoidal shape has a second base that is 5-25 mm long (e.g., 5, 6, 7, 8, 9, 10, 11, 12, 13, 14, 15, 16, 17, 18, 19, 20, 21, 22, 23, 24, or 25 mm). In some embodiments, the trapezoidal shape has a height of 2-15 mm (e.g., 2, 3, 4, 5, 6, 7, 8, 9, 10, 11, 12, 13, 14, or 15 mm). In some embodiments, the trapezoidal shape is an isosceles trapezoid. In some embodiments, the trapezoidal shape comprises two angles of 30 to 60 degrees (e.g., 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 degrees).

In some embodiments, the non-rectangular sample passage slot has a triangular shape. In some embodiments, the triangular shape has a base that is 20-40 mm long (e.g., 20, 21, 22, 23, 24, 25, 26, 27, 28, 29, 30, 31, 32, 33, 34, 35, 36, 37, 38, 39, or 40 mm). In some embodiments, the triangular shape has a height of 2-30 mm (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 mm). In some embodiments, the triangular shape is an isosceles triangle. In some embodiments, the triangular shape comprises base angles of 30 to 60 degrees (e.g., 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 degrees).

In some embodiments, the non-rectangular sample passage slot has a shape that is an arch, segment of a circle, or segment of an ellipse. In some embodiments, the assay device further comprises a lid configured to seal said chamber. In some embodiments, the lid engages the indication structure of the chamber to provide feedback to a user indicating that the lid is securely affixed to the chamber (e.g., to prevent the leakage of sample from the chamber). Accordingly, in some embodiments, the indication structure is configured to produce a vibration when said lid is securely engaged with said chamber. In some embodiments, the vibration provides an audio or haptic signal to a user of the assay device. In some embodiments, the lid is a screw-lid. In some embodiments, the lid is a pop-lid (e.g., is pushed onto the chamber). In some embodiments, the test device further comprises a sample validity testing strip configured to test for at least 3 (e.g., 3, 4, 5, 6, 7, 8 or more) sample characteristics (e.g., pH, specific gravity, presence of creatinine, and presence of an oxidant). In some embodiments, the assay device does not comprise a membrane (e.g., a raised polyester membrane) on the bottom wall of said assay device in contrast to previous devices.

In some embodiments, the assay device comprises components (e.g., chamber, reservoir, lid, and/or test device) made from a polymer. In some embodiments, the assay device comprises components made of a thermoplastic, a specialty plastic, a thermoset, and/or an engineering plastic. Thermoplastics include but are not limited to polyamideimide (PAD, polyethersulfone (PES), polyarylsulfone (PAS), polyetherimide (PEI), polyarylate (PAR), polysulfone (PSO), polyamide (PA), polycarbonate (PC), styrene-maleic anhydride (SMA), chlorinated PVC (CPVC), poly(methylmethacrylate) (PMMA), styrene-acrylonitrile (SAN), polystyrene (PS), acrylonitrile-butadiene-styrene (ABS), poly(ethyleneterephthalate) (PET), poly(vinylchloride) (PVC), polyetherketone (PEK), polyetheretherketone (PEEK), polytetrafluoroethylene (PTFE), poly(phenylene sulfide) (PPS), liquid crystal polymer (CCP), nylon-6,6, nylon-6, nylon-6,12, nylon-11, nylon 12, acetal resin, low and high density polypropylene (PP), high density polyethylene (HDPE), low density polyethylene (LDPE), polystyrene, ethylene-vinyl acetate, poly-vinyl-acetate, polyacrylic, etc., or a copolymer or a combination thereof. Specialty plastics include but are not limited to fluorocarbon polymers and infusible film products such as Kapton, Upilex polyimide film etc., a copolymer or a combination thereof. Thermosets include but not are limited to phenolics, epoxies, urea-formaldehyde, silicones, etc., a copolymer or a combination thereof. Engineering plastics include but are not limited to acetyl resins, polyamide, polyetherimides, polyesters, liquid crystal polymers, polycarbonate resins, poly(phenylene ether) alloys, polysulfone resins, polyamideimide resins, etc., a copolymer or a combination thereof. In some embodiments, the chamber, the reservoir, the lid, and/or the test device comprises high impact polystyrene.

In some embodiments, the test strips are configured to test for the presence, absence, amount, and/or concentration of drugs of abuse (e.g., a plurality of amphetamine, barbiturate, benzodiazepine, buprenorphine, cocaine, tetrahydrocannabinol, ethyl glucuronide, methadone, methamphetamine, 3,4-methylenedioxy-methamphetamine, opiate, oxycodone, phencyclidine, propoxyphene, 6-monoacetyl morphine, morphine, fentanyl, tramadol, synthetic cannabinoids (e.g., K2, spice, etc.), and/or ketamine). In some embodiments, the test strips are configured to test for at least 15 drugs of abuse. In some embodiments, quantitative lateral flow assays (e.g., to determine amount and/or concentration of an analyte) are employed. See, e.g., U.S. Pat. App. Pub. No. 2015/0293085 and Koczula and Gallotta (2016) “Lateral flow assays” Essays Biochem 60(1): 111-120, each of which is incorporated herein by reference.

In some embodiments, the assay device further comprises a sample (e.g., urine, blood, serum, or spinal fluid sample).

In some embodiments, the assay device further comprises a reservoir seal providing an airtight seal for said reservoir. In some embodiments, the reservoir seal, in cooperation with said non-rectangular specimen passage slot, controls fluid passage from said chamber to said reservoir. In some embodiments, the reservoir seal, in cooperation with said non-rectangular specimen passage slot, prevents excess influx of a sample into said reservoir upon introduction of said sample into said chamber.

In related embodiments, the technology provides methods for testing a sample for the presence of an analyte. For example, in some embodiments, methods comprise providing an assay device as described herein and providing a fluid sample in the chamber. In some embodiments, methods further comprise observing a detection zone of a test strip (e.g., to detect a result). In some embodiments, methods further comprise observing a sample validity testing strip. In some embodiments, the sample validity testing strip displays the results of 4 sample validity tests simultaneously (e.g., pH, specific gravity, presence of creatinine, and presence of an oxidant). In some embodiments, methods comprise sealing the chamber with a lid. In some embodiments, sealing the chamber with the lid causes an indication structure to produce a vibration (e.g., indicating that the lid sealed the chamber). In some embodiments, methods further comprise sensing an audio or haptic signal indicating that said lid sealed said chamber. In some embodiments, methods comprise removing a peel-off label to expose a detection zone. In some embodiments, methods comprise removing a peel-off label to expose a sample validity testing strip. In some embodiments, methods comprise reporting a result to a user. In some embodiments, methods comprise acquiring an image of said detection zone to detect a result. In some embodiments, methods comprise use of an optical device to detect a result.

In related embodiments, the technology provides an assay device system. In some embodiments, the assay device system comprises a body (e.g., comprising a chamber and a reservoir), a test device, and a plurality of test strips. In some embodiments, the assay device system further comprises a sample. In some embodiments, the assay device system further comprises an imaging component (e.g., an optical detector, an imaging device (e.g., a camera)). In some embodiments, the assay device system further comprises a reporting component (e.g., a printer, a software component to communicate results to another computer or user, a display, an audio component, a light (e.g., an LED)). In some embodiments, the assay device system further comprises a non-rectangular sample passage slot fluidly connecting said chamber and said reservoir. In some embodiments, the non-rectangular sample passage slot has a trapezoidal shape. In some embodiments, the trapezoidal shape has a first base that is 20-40 mm long (e.g., 20, 21, 22, 23, 24, 25, 26, 27, 28, 29, 30, 31, 32, 33, 34, 35, 36, 37, 38, 39, or 40 mm). In some embodiments, the trapezoidal shape has a second base that is 5-25 mm long (e.g., 5, 6, 7, 8, 9, 10, 11, 12, 13, 14, 15, 16, 17, 18, 19, 20, 21, 22, 23, 24, or 25 mm). In some embodiments, the trapezoidal shape has a height of 2-15 mm (e.g., 2, 3, 4, 5, 6, 7, 8, 9, 10, 11, 12, 13, 14, or 15 mm). In some embodiments, the trapezoidal shape is an isosceles trapezoid. In some embodiments, the trapezoidal shape comprises two angles of 30 to 60 degrees (e.g., 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 degrees). In some embodiments, the non-rectangular sample passage slot has a triangular shape. In some embodiments, the triangular shape has a base that is 20-40 mm long (e.g., 20, 21, 22, 23, 24, 25, 26, 27, 28, 29, 30, 31, 32, 33, 34, 35, 36, 37, 38, 39, or 40 mm). In some embodiments, the triangular shape has a height of 2-30 mm (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 mm). In some embodiments, the triangular shape is an isosceles triangle. In some embodiments, the triangular shape comprises base angles of 30 to 60 degrees (e.g., 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 degrees).

In some embodiments, the assay device system further comprises a lid configured to seal said chamber. In some embodiments of the assay device system, the chamber comprises an indication structure configured to produce a vibration when the lid is securely engaged with said chamber. In some embodiments, the assay device system further comprises a sample validity testing strip configured to test for at least 3 (e.g., 3, 4, 5, 6, 7, or 8) sample characteristics. In some embodiments, the assay device system does not comprise a membrane (e.g., a raised polyester membrane) on the bottom wall of said chamber or said reservoir. In some embodiments, the assay device system further comprises a reservoir seal providing an airtight seal for said reservoir.

In related embodiments, the technology provides methods of manufacturing an assay device. For example, in some embodiments, methods of manufacturing an assay device comprise producing a body from a polymer (e.g., a body comprising a reservoir in fluid communication with a chamber through a non-rectangular sample passage slot (e.g., wherein the chamber comprises an indication structure)); placing a test device (e.g., comprising a polymer test device panel, a test strip (e.g., 1, 2, 3, 4, 5, 6, 7, 8, 9, 10, 11, 12, 13, 14, 15, or more test strips), and a sample validity testing strip) into the reservoir; and sealing the reservoir with a reservoir seal. In some embodiments, the non-rectangular sample passage slot has a trapezoidal shape. In some embodiments, the trapezoidal shape has a first base that is 20-40 mm long (e.g., 20, 21, 22, 23, 24, 25, 26, 27, 28, 29, 30, 31, 32, 33, 34, 35, 36, 37, 38, 39, or 40 mm). In some embodiments, the trapezoidal shape has a second base that is 5-25 mm long (e.g., 5, 6, 7, 8, 9, 10, 11, 12, 13, 14, 15, 16, 17, 18, 19, 20, 21, 22, 23, 24, or 25 mm). In some embodiments, the trapezoidal shape has a height of 2-15 mm (e.g., 2, 3, 4, 5, 6, 7, 8, 9, 10, 11, 12, 13, 14, or 15 mm). In some embodiments, the trapezoidal shape is an isosceles trapezoid. In some embodiments, the trapezoidal shape comprises two angles of 30 to 60 degrees (e.g., 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 degrees). In some embodiments, the non-rectangular sample passage slot has a triangular shape. In some embodiments, the triangular shape has a base that is 20-40 mm long (e.g., 20, 21, 22, 23, 24, 25, 26, 27, 28, 29, 30, 31, 32, 33, 34, 35, 36, 37, 38, 39, or 40 mm). In some embodiments, the triangular shape has a height of 2-30 mm (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 mm). In some embodiments, the triangular shape is an isosceles triangle. In some embodiments, the triangular shape comprises base angles of 30 to 60 degrees (e.g., 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 degrees). In some embodiments, methods of manufacturing an assay device further comprise producing a lid configured to seal said chamber. In some embodiments, methods of manufacturing an assay device comprise producing the test device panel. In some embodiments, methods of manufacturing an assay device comprise producing the test strips. In some embodiments, methods of manufacturing an assay device produce a test device panel comprising a channel or a plurality of channels (e.g., 2, 3, 4, 5, 6, 7, 8, 9, 10, 11, 12, 13, 14, 15, or more channels) and the methods further comprise placing a test strip into a channel and/or placing the test strips into the channels. In some embodiments, producing the body in methods of manufacturing an assay device comprises use of a technology such as, e.g., injection molding, machining, or three-dimensional printing. In some embodiments, producing the test device panel comprises use of a technology such as, e.g., injection molding, machining, or three-dimensional printing.

In some embodiments, the technology provides methods of manufacturing an assay device and/or one or more components of an assay device (e.g., chamber, reservoir, lid, and/or test device) from a polymer. In some embodiments, the technology provides methods of manufacturing an assay device and/or one or more components of an assay device (e.g., chamber, reservoir, lid, and/or test device) from a thermoplastic, a specialty plastic, a thermoset, and/or an engineering plastic. Thermoplastics include but are not limited to polyamideimide (PAD, polyethersulfone (PES), polyarylsulfone (PAS), polyetherimide (PEI), polyarylate (PAR), polysulfone (PSO), polyamide (PA), polycarbonate (PC), styrene-maleic anhydride (SMA), chlorinated PVC (CPVC), poly(methylmethacrylate) (PMMA), styrene-acrylonitrile (SAN), polystyrene (PS), acrylonitrile-butadiene-styrene (ABS), poly(ethyleneterephthalate) (PET), poly(vinylchloride) (PVC), polyetherketone (PEK), polyetheretherketone (PEEK), polytetrafluoroethylene (PTFE), poly(phenylene sulfide) (PPS), liquid crystal polymer (CCP), nylon-6,6, nylon-6, nylon-6,12, nylon-11, nylon 12, acetal resin, low and high density polypropylene (PP), high density polyethylene (HDPE), low density polyethylene (LDPE), polystyrene, ethylene-vinyl acetate, poly-vinyl-acetate, polyacrylic, etc., or a copolymer or a combination thereof. Specialty plastics include but are not limited to fluorocarbon polymers and infusible film products such as Kapton, Upilex polyimide film etc., a copolymer or a combination thereof. Thermosets include but not are limited to phenolics, epoxies, urea-formaldehyde, silicones, etc., a copolymer or a combination thereof. Engineering plastics include but are not limited to acetyl resins, polyamide, polyetherimides, polyesters, liquid crystal polymers, polycarbonate resins, poly(phenylene ether) alloys, polysulfone resins, polyamideimide resins, etc., a copolymer or a combination thereof. In some embodiments, the chamber, the reservoir, the lid, and/or the test device comprises high impact polystyrene.

In particular embodiments, the technology provides a method of manufacturing an assay device in which the actions of a human are minimized and/or eliminated. Accordingly, in some embodiments, producing the body is automated, placing the test device into said reservoir is automated, and/or sealing the reservoir with the reservoir seal is automated. In some embodiments, methods of manufacturing an assay device further comprise affixing a label to said body (e.g., using automation).

The technology is not limited in the analytes detected and/or its uses. For example, in some embodiments, the technology provides use of an assay device as described herein to test a sample for the presence, absence, concentration, and/or amount of an analyte. In some embodiments, the technology provides use of an assay device as described herein to test a sample for the presence, absence, concentration, and/or amount of a drug of abuse.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1A is an exploded view drawing of an embodiment of the assay device 100 described herein. In the embodiment of the assay device 100 shown in FIG. 1A, the assay device 100 comprises a body 200 that comprises a chamber 210 and a reservoir 220. The reservoir 220 is configured to hold a test device panel 400 and a reservoir seal 500 is configured to provide an airtight seal for the reservoir 220. A chamber seal 300 is configured to seal the chamber 210 and the chamber seal comprises a chamber seal indication structure 310.

FIG. 1B is an exploded view drawing of an embodiment of the assay device 100 described herein. In the embodiment of the assay device 100 shown in FIG. 1B, the assay device 100 comprises a chamber (e.g., as shown in FIG. 1A as chamber 210) that comprises an upper opening 211, a side wall 212, and a bottom wall 213. The reservoir (e.g., as shown in FIG. 1A as reservoir 220) comprises a reservoir opening 221. The chamber further comprises threads 215 configured to rotatably mate with threads 320 on the chamber seal 300. In some embodiments, the chamber comprises a chamber indication structure 216.

FIG. 2A is a top view of an embodiment of the body of an assay device described herein. The body comprises a chamber 210 and a reservoir 220. The sample passage slot wall 214 comprises a chamber indication structure 216. Dimensions of the body of the assay device are indicated by a and b, which are discussed herein. The cross-section view along the line A-A is shown in FIG. 2F.

FIG. 2B is a bottom view of an embodiment of the body of an assay device described herein.

FIG. 2C is a front view of an embodiment of the body of an assay device described herein. The height of the body of the assay device is indicated by h, which is discussed herein. The cross-section view along the line B-B is shown in FIG. 2G.

FIG. 2D is a left view of an embodiment of the body of an assay device described herein.

FIG. 2E is a right view of an embodiment of the body of an assay device described herein.

FIG. 2F is a cross-section view (along the line A-A shown in FIG. 2A) of an embodiment of the body of an assay device described herein. FIG. 2F shows a body comprising a chamber 210, a reservoir 220, a bottom wall 213, and a sample passage slot wall 214. A diameter of the body of the assay device is indicated by a, which is discussed herein.

FIG. 2G is a cross-section view (along the line B-B shown in FIG. 2C) of an embodiment of the body of an assay device as described herein. The cross-section view shows the sample passage slot wall 214 and the bottom wall 213. The sample passage slot wall 214 comprises a sample passage slot 230 (e.g., a non-rectangular sample passage slot (e.g., a trapezoidal sample passage slot (e.g., comprising a top side of length a, a bottom side of length b, and a height of c))), an umbrella structure 240, a hole 250 (e.g., comprising a diameter of d), and a chamber indication structure 216.

FIG. 2H is a drawing of an embodiment of a trapezoidal sample passage slot 230. In some embodiments, the trapezoidal sample passage slot 230 comprises a first (e.g., bottom) base 231, a second (e.g., top) base 232, a first leg 233, a second leg 234, a first bottom angle 235, a second bottom angle 236, a first top angle 237, and a second top angle 238. In some embodiments, the trapezoidal passage slot 230 comprises a first vertical segment 239A and a second vertical segment 239B (e.g., the first vertical segment and second vertical segment clip portions of the trapezoid at the first bottom angle 235 and the second bottom angle 236).

FIG. 2I is a cross-section view (along the line B-B shown in FIG. 2C) of an embodiment of the body of an assay device described herein comprising a triangular sample passage slot 230.

FIG. 2J is a cross-section view of an embodiment of the body of an assay device described herein comprising a triangular sample passage slot and tilted at an angle of 30°. The direction of the gravity vector is shown by the arrow labeled with a “g”.

FIG. 2K is a cross-section view of an embodiment of the body of an assay device described herein comprising a trapezoidal sample passage slot and tilted at an angle of 30°. The direction of the gravity vector is shown by the arrow labeled with a “g”.

FIG. 3A is a three-dimensional view of an embodiment of a chamber seal 300 as provided herein comprising a chamber seal indication structure 310.

FIG. 3B is a top view of an embodiment of a chamber seal as provided herein.

FIG. 3C is a bottom view of an embodiment of a chamber seal as provided herein comprising a chamber seal indication structure 310.

FIG. 3D is a front view of an embodiment of a chamber seal as provided herein comprising a chamber seal indication structure 310. A dimension of the chamber seal is indicated by a, which is discussed herein. The cross-section view along the line A-A is shown in FIG. 3G.

FIG. 3E is a left view of an embodiment of a chamber seal as provided herein comprising threads 320.

FIG. 3F is a front view of an embodiment of a chamber seal as provided herein comprising a chamber seal indication structure 310 and comprising threads 320.

FIG. 3G is a cross-section view (along the line A-A shown in FIG. 3D) of an embodiment of a chamber seal as provided herein comprising a chamber seal ring 330 (e.g., an O-ring). Diameters of the chamber seal are indicated by a, b, and c, which are discussed herein.

FIG. 4A is a three-dimensional view of an embodiment of a test device panel 400 as described herein.

FIG. 4B is a top view of an embodiment of a test device panel as described herein.

FIG. 4C is a bottom view of an embodiment of a test device panel as described herein.

FIG. 4D is a front view of an embodiment of a test device panel as described herein comprising a plurality of channels 410, e.g., configured to hold test elements (e.g., test strips). Dimensions of the test device panel are indicated by a, b, and c, which are discussed herein.

FIG. 4E is a back view of an embodiment of a test device panel as described herein.

FIG. 4F is a left view of an embodiment of a test device panel as described herein. Dimensions of the test device panel are indicated by h and d, which are discussed herein.

FIG. 4G is a right view of an embodiment of a test device panel as described herein.

FIG. 4H is a front view of an embodiment of a test device panel as described herein comprising a plurality of channels 410, e.g., configured to hold test elements (e.g., test strips) and a center element 420 to facilitate migration of a fluid specimen into the test strips.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to detecting one or more analytes in a sample and particularly, but not exclusively, to devices, methods, systems, and kits for detecting one or more drugs-of-abuse analytes in a biological sample.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a sequencing step, etc.

As used herein, the word “presence” or “absence” (or, alternatively, “present or “absent”) is used in a relative sense to describe the amount or level of a particular entity (e.g., an analyte). For example, when an analyte is said to be “present” in a sample, it means the level or amount of the analyte is above a pre-determined threshold; conversely, when an analyte is said to be “absent” in a test sample, it means the level or amount of the analyte is below a pre-determined threshold. The pre-determined threshold may be the threshold for detectability associated with the particular assay used to detect the analyte or any other threshold. When an analyte is “detected” in a sample it is “present” in the sample; when an analyte is “not detected” it is “absent” from the sample. Further, a sample in which an analyte is “detected” or in which the analyte is “present” is a sample that is “positive” for the analyte. A sample in which an analyte is “not detected” or in which the analyte is “absent” is a sample that is “negative” for the analyte.

As used herein, an “increase” or a “decrease” refers to a detectable (e.g., measured) positive or negative change in the value of a variable relative to a previously measured value of the variable, relative to a pre-established value, and/or relative to a value of a standard control. In some embodiments, an increase is a positive change, preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change, preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, the term “system” denotes a set of components, real or abstract, comprising a whole where each component interacts with or is related to at least one other component within the whole.

As used herein, the term “assaying” refers to qualitatively or quantitatively testing a sample for an analyte (e.g., for the presence, absence, concentration, and/or amount of the analyte). Assaying may comprise an immunological test, a chemical test, an enzymatic test, and the like. In some embodiments, the present technology assays for the presence, absence, concentration, and/or amount of a variety of analytes such as but not limited to, a chemical, an organic compound, an inorganic compound, a metabolic product, a drug or a drug metabolite, an organism or a metabolite of such an organism, a nucleic acid, a protein, a hormone, or a combination thereof. Assaying may involve comparing the results obtained against a positive or negative control as is common in the biochemical and immunological arts. When determining the concentration of an analyte, the assay may also include at least one quantitative control to determine the amount of analyte present and may further include mathematical calculations such as comparing the amount of analyte to the volume within the collection container or reservoir.

As used herein, an element of the present technology is “integral” to another element of the present technology when the two elements are manufactured or assembled as a single piece.

As used herein, an element of the present technology is “separate” from another element of the present technology when the two elements are manufactured or provided as separate pieces.

As used herein, the term “reagent” refers to a composition, e.g., comprising a chemical (e.g., organic compounds and inorganic compounds), enzymes, and combinations thereof. A reagent can be provided in gaseous, solid, or liquid form, or any combination thereof, and can be a component of a solution or suspension. In some embodiments, a reagent includes fluids useful in methods of detecting analytes in a sample, such as buffers, anticoagulants, diluents, test reagents, specific binding members, detectable labels, enzymes, and the like. A reagent can also include an extractant, such as a buffer or chemical, to extract an analyte from a sample or a sample collection device. For example, a buffer can be used to extract analytes from a sample or specimen.

As used herein, the term “detection device”, “assaying device”, and “assay device” are used interchangeably to refer to a device for detecting the presence, absence, concentration, and/or amount of an analyte in a sample or specimen. In some embodiments, an “assay device” is a “test device”; in some embodiments, an “assay device” comprises a “test device” and additional components. Assay devices of the present technology include but are not limited to lateral flow detection devices (e.g., assay strip devices) and columns. In lateral flow detection devices, the liquid sample or specimen moves through a matrix or material by lateral flow or capillary action. An exemplary lateral flow test device is an immuno-chromatographic device. In a typical immunochromatographic device the sample moves through a sample application zone, a reagent zone, and a detection zone. The sample application zone is a region of the lateral flow detection device that is contacted first by the sample; the reagent zone is a region in which particular reagents for the desired assay are positioned such that they migrate with an analyte along the device; and the detection zone is a region in which the results of the assay are visualized, presented, and/or determined. In some embodiments, a mobilizable reagent such as a labeled antibody is provided in the reagent zone and an immobilized reagent is provided in the detection zone. A lateral flow detection device may be used in a substantially vertical or a substantially horizontal orientation or in an orientation substantially between vertical and horizontal. Preferably, neither a reagent zone nor a detection zone contacts the sample or analyte unless the sample or analyte migrates along the lateral flow detection device. Persons knowledgeable in the art commonly refer to a lateral flow detection device using terms such as “immunochromatographic”, “dip stick”, “membrane technology”, and “test strip.”

As used herein, the term “analyte” refers to a compound or composition to be detected or measured. An analyte is generally capable of binding to a ligand, a receptor, or an enzyme. The analyte may be an antibody or antigen such as a protein or drug, or a metabolite. The precise nature of antigenic and drug analytes together with numerous examples thereof are disclosed in U.S. Pat. No. 4,299,916, particularly columns 16 to 23, and in U.S. Pat. No. 4,275,149, particularly columns 17 and 18, each of which is incorporated herein by reference. Analytes can include antibodies and receptors, including active fragments or fragments thereof. An analyte can include an analyte analogue, which is a derivative of an analyte, such as, for example, an analyte altered by chemical or biological methods, such as by the action of reactive chemicals, such as adulterants or enzymatic activity. An analyte can be but is not limited to a drug, a drug of abuse, a hormone, a protein, a nucleic acid, an element, an ion, a small molecule (e.g., a natural or synthetic small molecule), an etiological agent, or a specific binding member.

As used herein, the term “antibody” refers to an immunoglobulin, or derivative or active fragment thereof, having an area on the surface or in a cavity, that specifically binds to a particular spatial and/or polar organization of another molecule. As used herein, the term “antibody” is used in its broadest sense to refer to whole antibodies, monoclonal antibodies (including human, humanized, or chimeric antibodies), polyclonal antibodies, and antibody fragments that can bind antigen (e.g., Fab′, F′ (ab)₂, Fv, single chain antibodies), comprising complementarity determining regions (CDRs) of the foregoing as long as they exhibit the desired biological activity. Antibodies encompassed by the present technology include but are not limited to IgG, IgM, and IgE; antibody light chains (e.g., kappa and lambda), antibody heavy chain fragments, Fc, and the like. The antibody can be prepared by techniques that are well known in the art such as, for example, immunization of a host and collection of sera or hybrid cell line technology. Determining the proper antibody may be performed by performing binding assays known in the immunological arts such as an ELISA with the analyte of interest.

As used herein, “antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995), incorporated herein by reference); single-chain antibody molecules; and multispecific antibodies formed from antibody fragments.

An antibody that “specifically binds to” or is “specific for” a particular antigen or an epitope on a particular antigen is one that binds to that particular antigen or epitope on a particular antigen without substantially binding to any other polypeptide or polypeptide epitope.

As used herein, the term “sample” or “specimen” refers to any material to be assayed for the presence, absence, concentration, and/or amount of an analyte. Preferably, a sample is a fluid sample such as a liquid sample. Examples of liquid samples that may be assayed include bodily fluids (e.g., blood, serum, plasma, saliva, urine, ocular fluid, semen, sputum, sweat, tears, and spinal fluid), water samples (e.g., samples of water from oceans, seas, lakes, rivers, and the like), samples from home, municipal, or industrial water sources, runoff water, or sewage samples; and food samples (e.g., milk, beer, juice, or wine). Viscous liquid, semisolid, or solid specimens may be used to create liquid solutions, eluates, suspensions, or extracts that can be samples. For example, throat or genital swabs may be suspended in a liquid solution to make a sample. Samples can include a combination of liquids, solids, gasses, or any combination thereof (e.g., a suspension of lysed or unlysed cells in a buffer or solution). Samples can comprise biological materials, such as cells, microbes, organelles, and biochemical complexes. Liquid samples can be made from solid, semisolid, or highly viscous materials, such as soils, fecal matter, tissues, organs, biological fluids, or other samples that are not fluid in nature. For example, solid or semisolid samples can be mixed with an appropriate solution, such as a buffer, a diluent, and/or extraction buffer. The sample can be macerated, frozen and thawed, or otherwise extracted to form a fluid sample. Residual particulates may be removed or reduced using conventional methods, such as filtration or centrifugation.

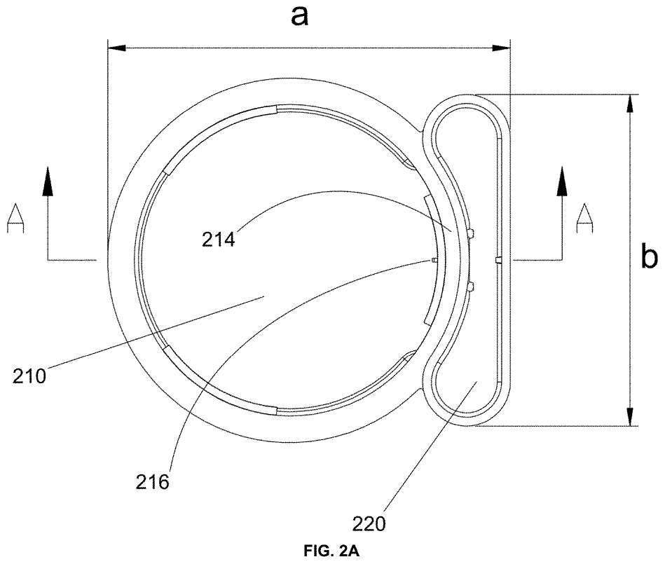

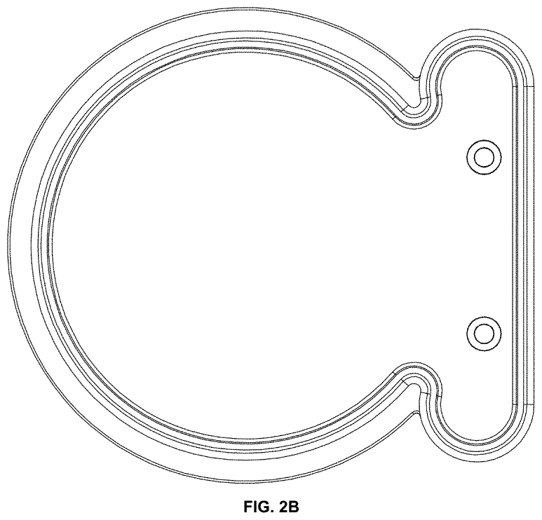

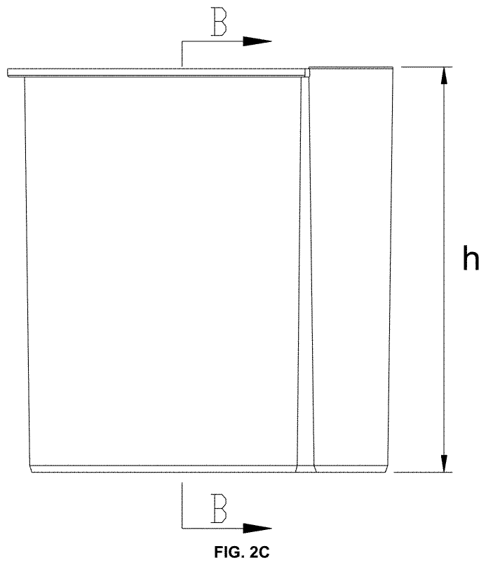

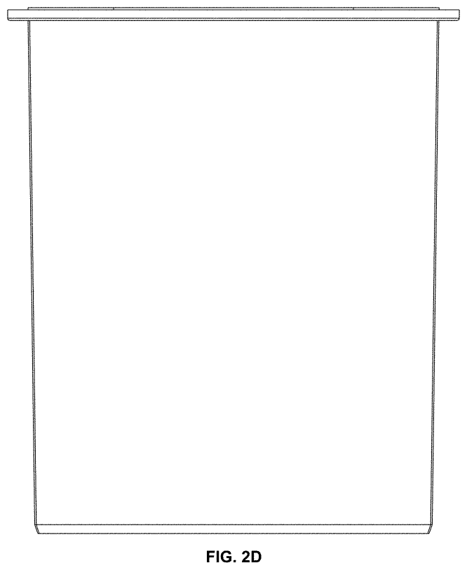

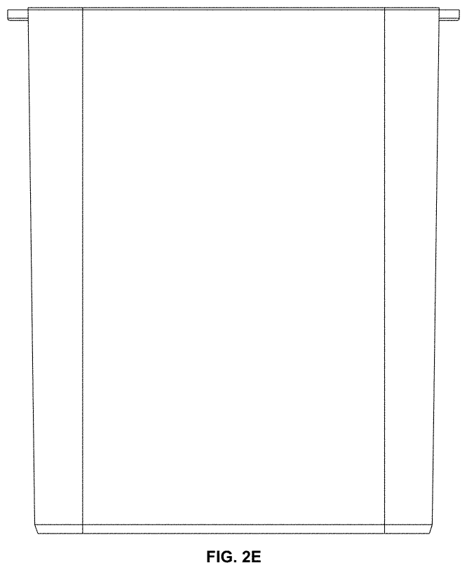

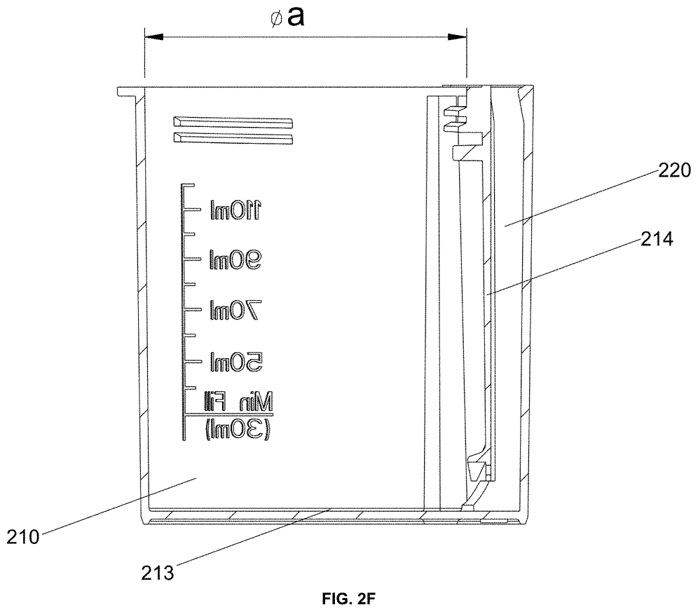

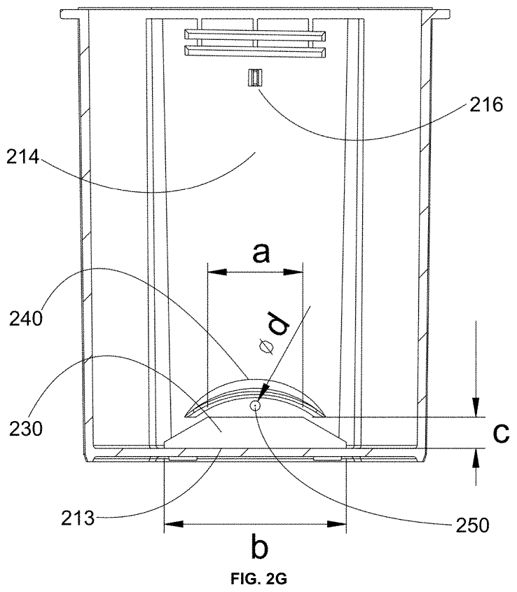

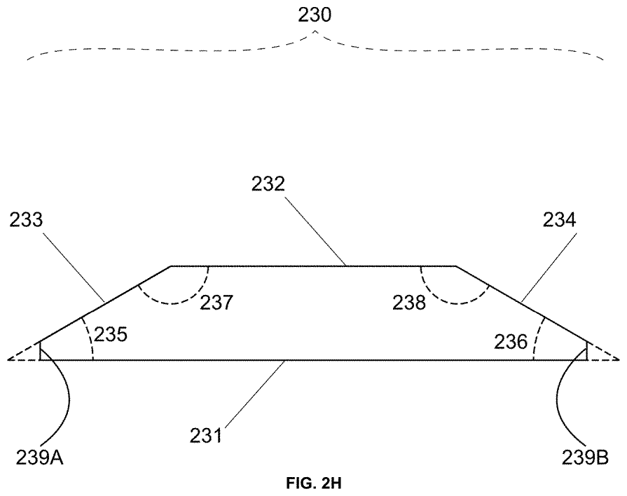

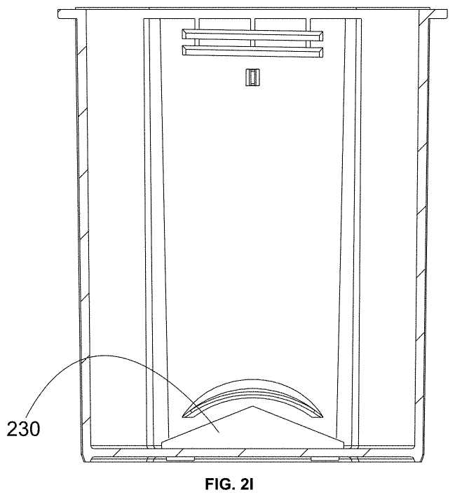

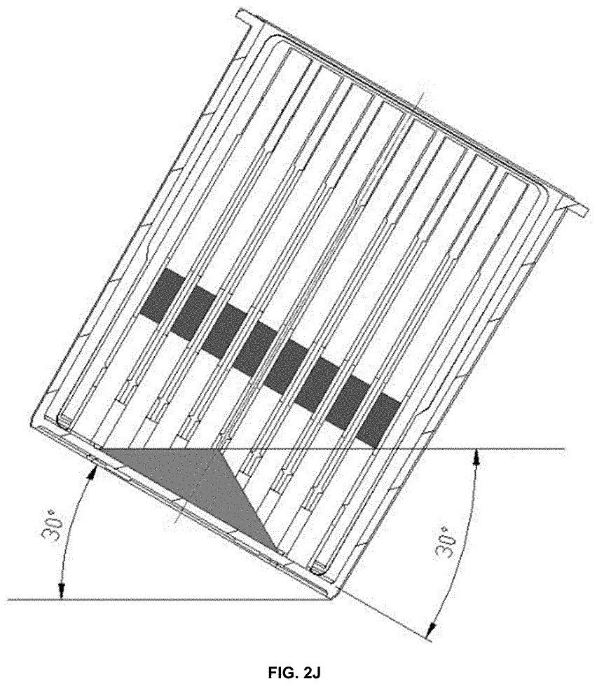

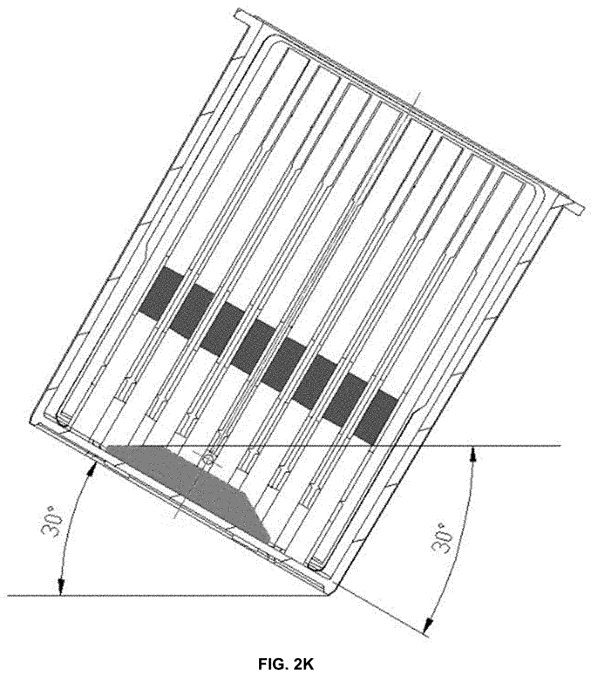

As used herein, the term “trapezoidal” refers to a shape that is or that is similar to a trapezoid. It is known in the art that a trapezoid is a convex quadrilateral with at least one pair of parallel sides. It is known in the art that the parallel (or substantially parallel) sides of a trapezoid are called the “bases” of the trapezoid and the other two sides are called the “legs” or the “lateral sides” of the trapezoid. In some embodiments, “trapezoidal” refers to the shape as shown in FIG. 2G and FIG. 2H as feature 230. While the shape of feature 230 in FIG. 2G and FIG. 2H is not strictly trapezoidal as understood in the geometry arts because it comprises two short vertical segments (e.g., 239A and 239B) connecting the bottom base 231 and the legs 233 and 234, one of ordinary skill in the art would understand that this shape approximates a trapezoid and is thus “trapezoidal”.

As used herein, the term “triangular” refers to a shape that is or that is similar to a triangle. It is known in the art that a triangle is a convex polygon with three sides. In some embodiments, “triangular” refers to the shape as shown in FIG. 2J and in FIG. 2I as feature 230. While the shape of feature 230 in FIG. 2I is not strictly triangular as understood in the geometry arts because it comprises two short vertical segments connecting the bottom base to two other sides, one of ordinary skill in the art would understand that this shape approximates a triangle and is thus “triangular”.

### DESCRIPTION

Provided herein is technology relating to detecting one or more analytes in a sample and particularly, but not exclusively, to devices, methods, systems, and kits for detecting one or more drugs-of-abuse analytes in a biological sample.

Assay Device

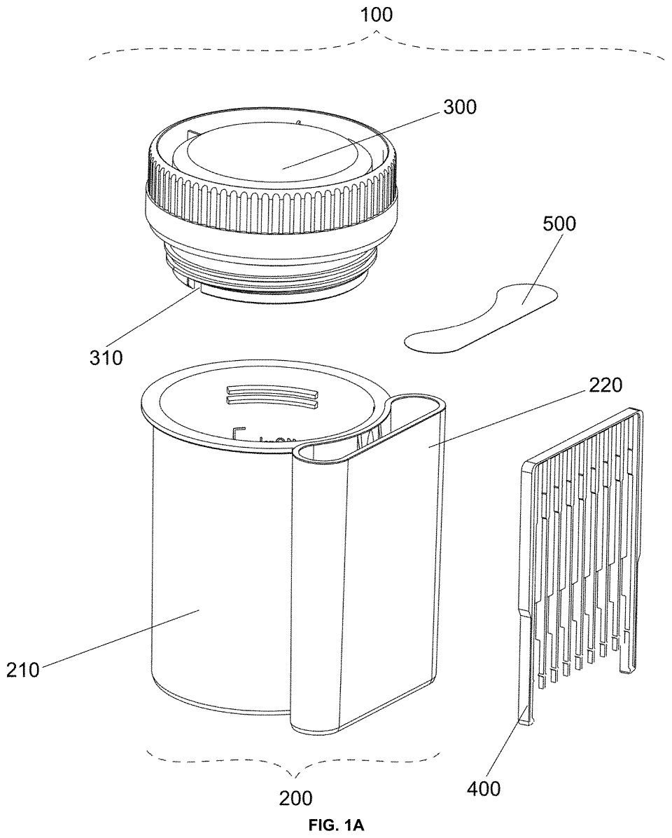

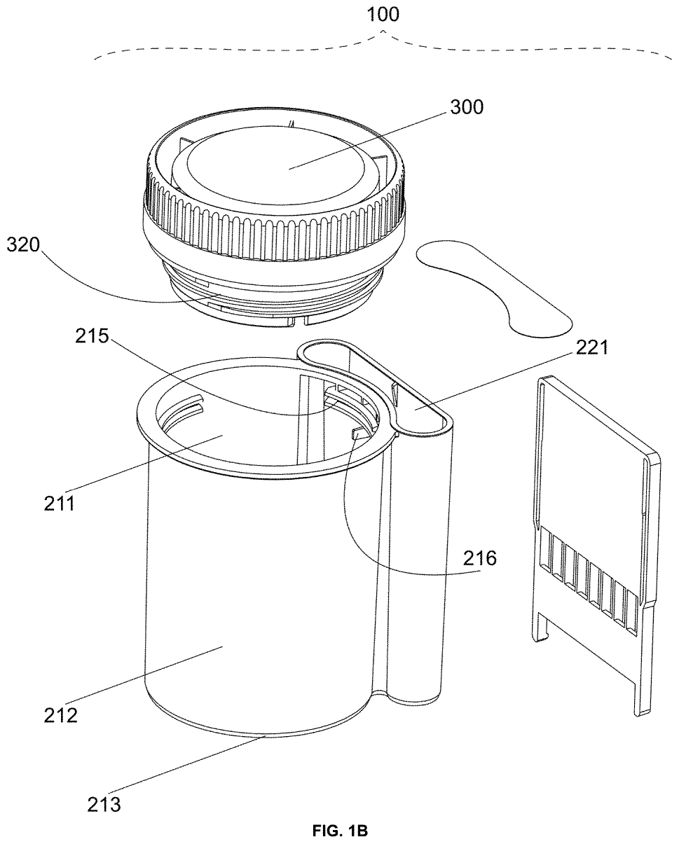

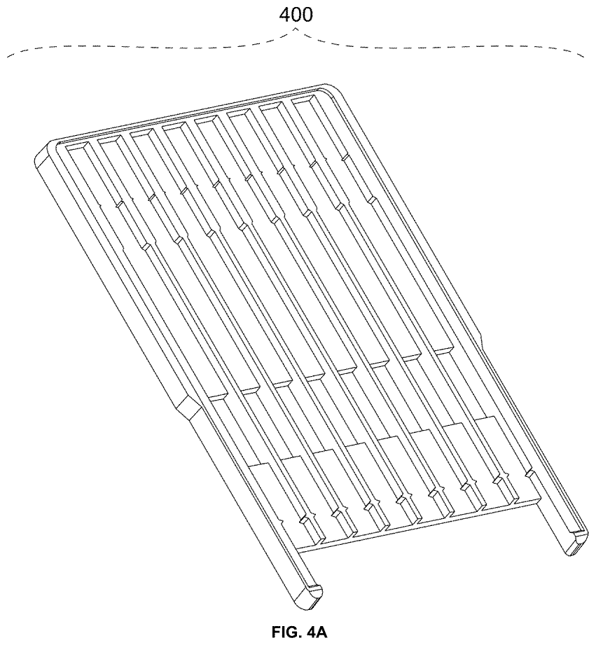

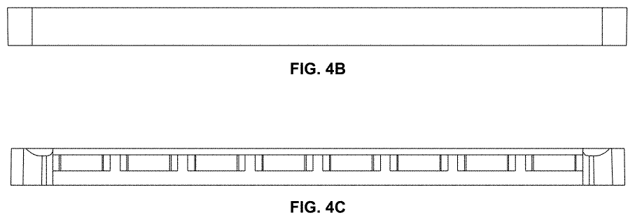

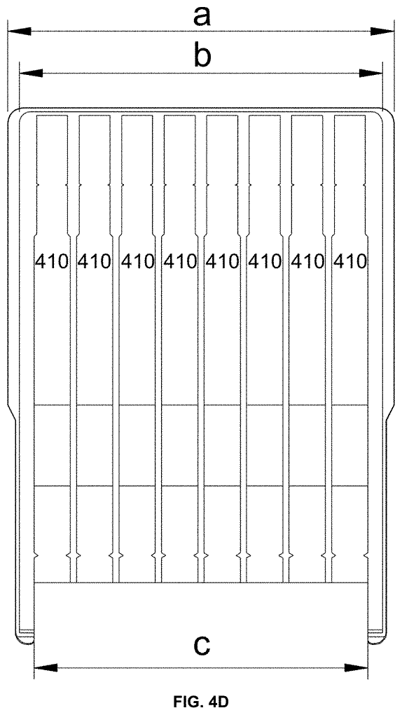

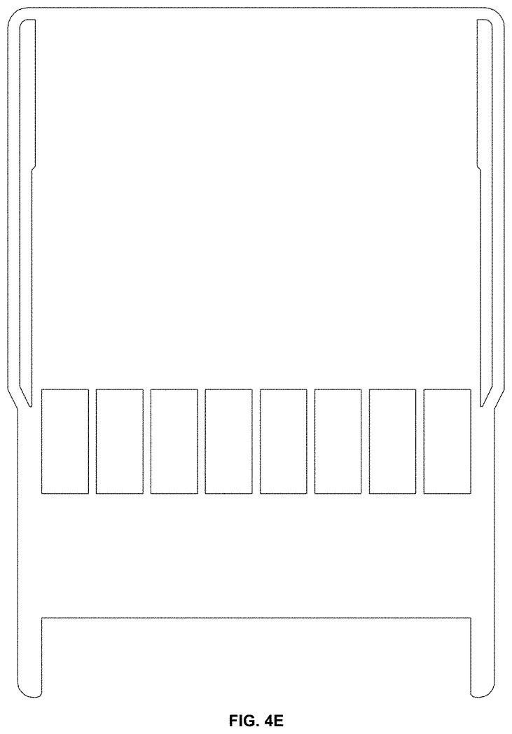

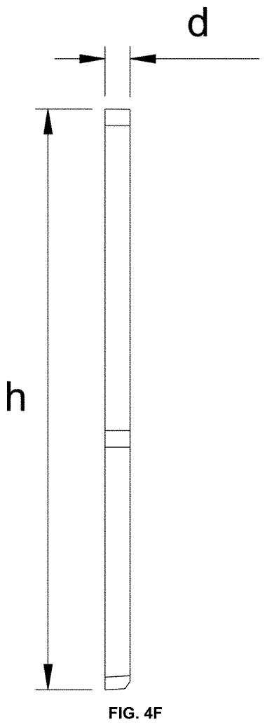

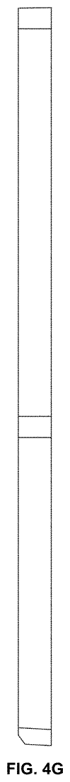

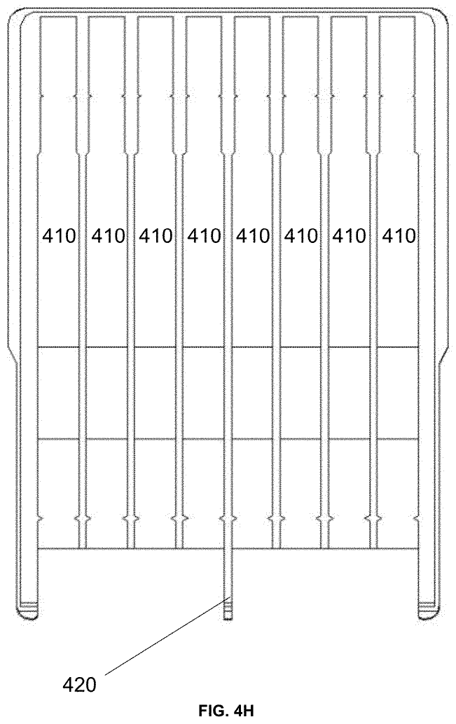

The technology relates to an assay device 100 (FIG. 1A and FIG. 1B). As provided by embodiments of the technology described herein, the assay device comprises a body 200. The body 200 (FIG. 2A to FIG. 2G and FIG. 2I) comprises a chamber 210 and a reservoir 220. The reservoir 220 comprises a test device (e.g., comprising a test device panel 400 (FIG. 4A to FIG. 4H) and one or more test strips). The body comprises a sample passage slot 230 fluidly connecting the chamber 210 and the reservoir 220 and configured to transfer at least a portion of a sample present in the chamber 210 to the reservoir 220. In some embodiments, the body 200 comprises an “umbrella” feature 240 that shields the test device (e.g., comprising a test device panel 400 and one or more test strips) from sample that may splash upon the test device upon providing a sample into the chamber 210. The umbrella 240 is above the sample passage slot 230. In some embodiments, the body comprises a vent hole 250. The vent hole 250 is above the sample passage slot 230. See FIG. 2G.

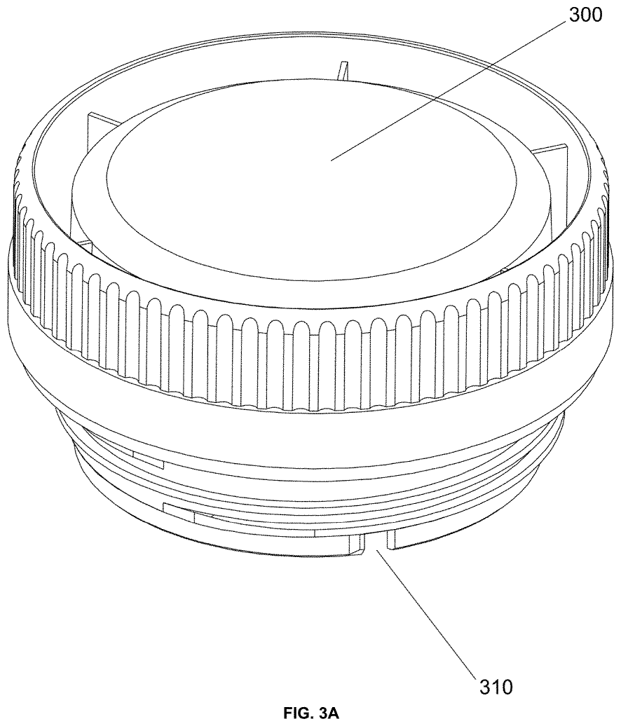

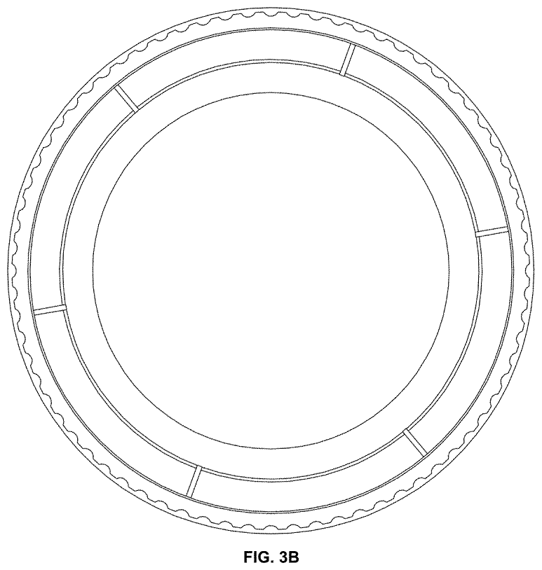

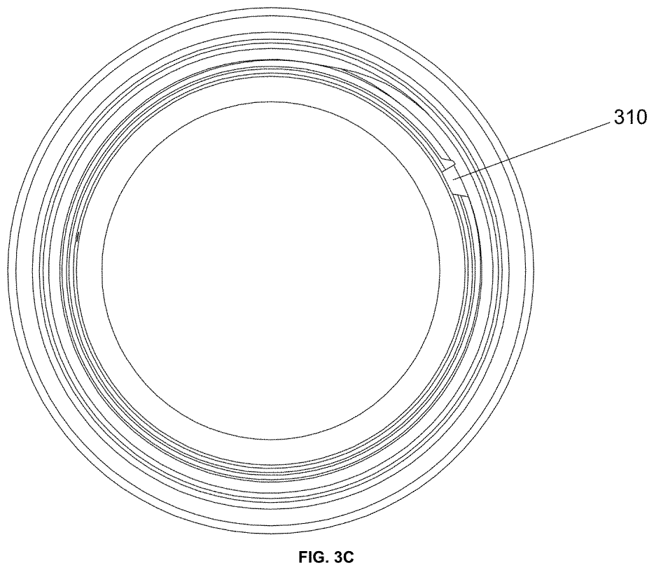

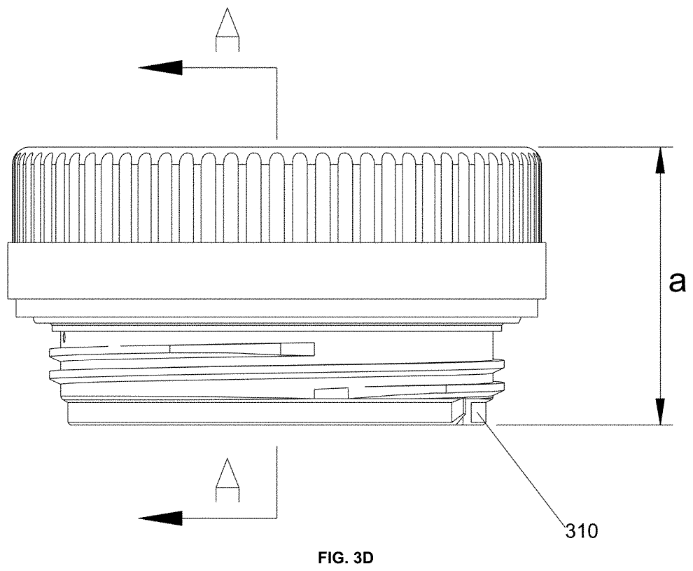

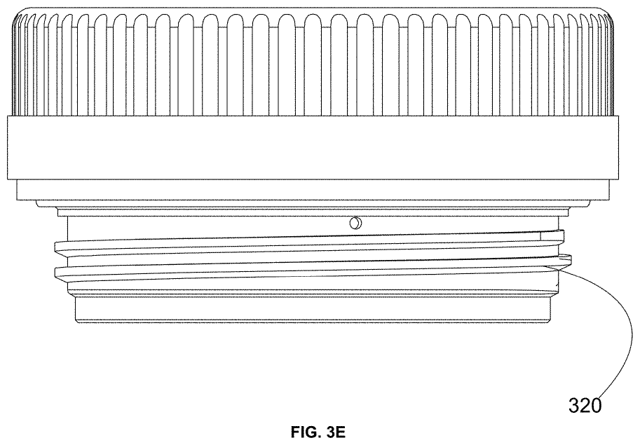

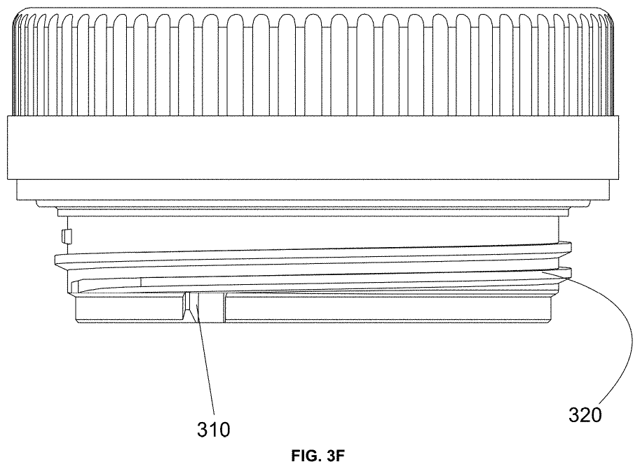

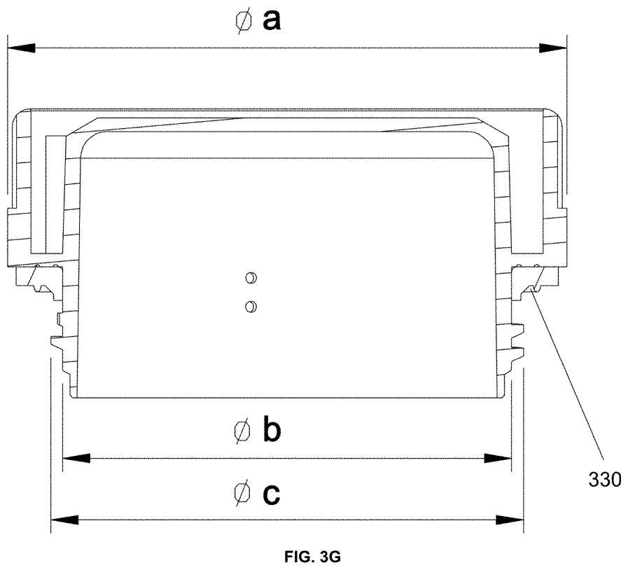

In some embodiments, a “body” (e.g., a body 200) comprises both the chamber 210 and reservoir 220 (and, optionally, components comprised by the chamber 210 and/or reservoir 220). In some embodiments, the assay device 100 also comprises a reservoir seal 500 that provides downward forces generated by air or gasses that compress upon entry of the sample to the reservoir 220, thus controlling the amount of sample that flows into the reservoir 220. In some embodiments, the assay device also comprises a chamber seal 300 (FIG. 3A to FIG. 3G) (e.g., a screw-lid, a snap-lid, etc.) comprising an indication structure 310 configured to produce a vibration (e.g., an audible sound and/or a haptic feedback) when the chamber seal 300 securely seals the chamber 210. See, e.g., U.S. Pat. No. 9,730,646, incorporated herein by reference (see, e.g., in particular the “cover body” 300 and the “indication structure element of the cover body” 302 referenced therein).

In some embodiments, the dimension a shown in FIG. 2A is approximately 60 to 80 mm (e.g., 60, 61, 62, 63, 64, 65, 66, 67, 68, 69, 70, 71, 72, 73, 74, 75, 76, 77, 78, 79, or 80 mm (e.g., 68.50 mm)). In some embodiments, the dimension b shown in FIG. 2A is approximately 45 to 65 mm (e.g., 45, 46, 47, 48, 49, 50, 51, 52, 53, 54, 55, 56, 57, 58, 59, 60, 61, 62, 63, 64, or 65 mm (e.g., 56.40 mm)). In some embodiments, the dimension h shown in FIG. 2C is approximately 60 to 80 mm (e.g., e.g., 60, 61, 62, 63, 64, 65, 66, 67, 68, 69, 70, 71, 72, 73, 74, 75, 76, 77, 78, 79, or 80 mm (e.g., 72.30 mm)). In some embodiments, the diameter a shown in FIG. 2F is approximately 45 to 65 mm (e.g., 45, 46, 47, 48, 49, 50, 51, 52, 53, 54, 55, 56, 57, 58, 59, 60, 61, 62, 63, 64, or 65 mm (e.g., 53.00 mm)). In some embodiments, the dimension a shown in FIG. 2G of the sample passage slot is approximately 10 to 20 mm (e.g., 10, 11, 12, 13, 14, 15, 16, 17, 18, 19, or 20 mm (e.g., 15.10 mm)). In some embodiments, the dimension b shown in FIG. 2G of the sample passage slot is approximately 20 to 30 mm (e.g., 20, 21, 22, 23, 24, 25, 26, 27, 28, 29, or 30 mm (e.g., 29.00 mm)). In some embodiments, the dimension c shown in FIG. 2G of the sample passage slot is approximately 1 to 10 mm (e.g., 1, 2, 3, 4, 5, 6, 7, 8, 9, or 10 mm (e.g., 5.00 mm)). In some embodiments, the diameter d shown in FIG. 2G of the vent hole is approximately 0.5 to 3 mm (e.g., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mm (e.g., 1.60 mm)).

In some embodiments, the dimension a shown in FIG. 3D is approximately 20 to 40 mm (e.g., 20, 21, 22, 23, 24, 25, 26, 27, 28, 29, 30, 31, 32, 33, 34, 35, 36, 37, 38, 39, or 40 mm (e.g., 32.00 mm)). In some embodiments, the diameter a shown in FIG. 3G is approximately 50 to 70 mm (e.g., 50, 51, 52, 53, 54, 55, 56, 57, 58, 59, 60, 61, 62, 63, 64, 65, 66, 67, 68, 69, or 70 mm (e.g., 62.00 mm)). In some embodiments, the diameter b shown in FIG. 3G is approximately 40 to 60 mm (e.g., 40, 41, 42, 43, 44, 45, 46, 47, 48, 49, 50, 51, 52, 53, 54, 55, 56, 57, 58, 59, or 60 mm (e.g., 49.80 mm)). In some embodiments, the diameter c shown in FIG. 3G is approximately 45 to 65 mm (e.g., 45, 46, 47, 48, 49, 50, 51, 52, 53, 54, 55, 56, 57, 58, 59, 60, 61, 62, 63, 64, or 65 mm (e.g., 52.40 mm)).

In some embodiments, the assay device 100 comprises a label or other scribe-able or scribed surface or surfaces on the chamber 210 or reservoir 220 on which to print, write, or display information. In some embodiments, a label is affixed to an outside surface of the chamber 210 or reservoir 220 by gluing, imprinting, texturing, scribing, etching, surface treating, impregnating, painting, screen printing, dyeing, coloring, embossing, or other suitable method. In some embodiments, a label is affixed to an inside surface of the chamber 210 or reservoir 220 by gluing, imprinting, texturing, scribing, etching, surface treating, impregnating, painting, screen printing, dyeing, coloring, embossing, or other suitable method. In some embodiments, a self-adhesive pre-printed label is affixed to the outer wall of the chamber 210 or reservoir 220. In some embodiments, a self-adhesive pre-printed label is affixed to the outer wall of the reservoir 220 and covers the portion of the test strips displaying test results. In some embodiments, the assay device 100 comprises a peel-off label covering the portion of the test strips displaying the test results and a designated operator removes the peel-off label to reveal the portion of the test strips displaying the test results and/or the sample validity testing strip.

In some embodiments, the assay device described herein is constructed using methods of construction known in the mechanical arts or medical device construction arts. The materials from which the assay device is manufactured are varied. In some embodiments, the assay device comprises a material that is metal, silicon, glass, ceramic, plastic, and synthetic and natural polymers and combinations and mixtures thereof. In some embodiments, the assay device comprises components (e.g., chamber, reservoir, lid, and/or test device) made from a polymer. In some embodiments, the assay device comprises components made of a thermoplastic, a specialty plastic, a thermoset, and/or an engineering plastic. Thermoplastics include but are not limited to polyamideimide (PAI), polyethersulfone (PES), polyarylsulfone (PAS), polyetherimide (PEI), polyarylate (PAR), polysulfone (PSO), polyamide (PA), polycarbonate (PC), styrene-maleic anhydride (SMA), chlorinated PVC (CPVC), poly(methylmethacrylate) (PMMA), styrene-acrylonitrile (SAN), polystyrene (PS), acrylonitrile-butadiene-styrene (ABS), poly(ethyleneterephthalate) (PET), poly(vinylchloride) (PVC), polyetherketone (PEK), polyetheretherketone (PEEK), polytetrafluoroethylene (PTFE), poly(phenylene sulfide) (PPS), liquid crystal polymer (CCP), nylon-6,6, nylon-6, nylon-6,12, nylon-11, nylon 12, acetal resin, low and high density polypropylene (PP), high density polyethylene (HDPE), low density polyethylene (LDPE), polystyrene, ethylene-vinyl acetate, poly-vinyl-acetate, polyacrylic, etc., or a copolymer or a combination thereof. Specialty plastics include but are not limited to fluorocarbon polymers and infusible film products such as Kapton, Upilex polyimide film etc., a copolymer or a combination thereof. Thermosets include but not are limited to phenolics, epoxies, urea-formaldehyde, silicones, etc., a copolymer or a combination thereof. Engineering plastics include but are not limited to acetyl resins, polyamide, polyetherimides, polyesters, liquid crystal polymers, polycarbonate resins, poly(phenylene ether) alloys, polysulfone resins, polyamideimide resins, etc., a copolymer or a combination thereof. In some embodiments, the chamber, the reservoir, the lid, and/or the test device comprises high impact polystyrene. In some embodiments, the assay device comprises a polypropylene and/or high impact polystyrene composition using an appropriate manufacturing method (e.g., pressure injection molding, machining, three-dimensional printing, etc.) In some embodiments, the assay device or a component thereof is constructed using other suitable methods of manufacturing such as milling, casting, blowing, spinning, three-dimensional printing, and other methods known in the mechanical arts and medical device construction.

In some embodiments, the technology provides an assay device 100 comprising a body 200 comprising an integrated chamber 210 and reservoir 220 that does not comprise a membrane (e.g., a raised polyester membrane) on its bottom (“floor”) wall 213 (e.g., in some embodiments the assay device comprises a “membrane-free” body 200, chamber 210, and/or reservoir 220); a chamber seal 300 (e.g., a lid) configured to seal the chamber 210 and comprising an indication structure 310 to provide audio and/or haptic feedback to a user that the chamber 210 is sealed by the chamber seal 300 (e.g., lid); a trapezoidal or arched sample passage slot 230; a reservoir seal 500 that limits the flow of sample from the chamber 210 to the reservoir 220; and a test device (e.g., comprising a test device panel 400 and one or more test strips) provided within the reservoir and comprising 6 or more (e.g., 6, 7, 8, 9, 10, 11, 12, 13, 14, 15, or more) 3.5-mm wide test strips for testing for 14 or more drugs of abuse (e.g., selected from amphetamine, barbiturate, benzodiazepine, buprenorphine, cocaine, tetrahydrocannabinol (THC), ethyl glucuronide, methadone, methamphetamine, 3,4-methylenedioxy-methamphetamine (MDMA), opiate, oxycodone, phencyclidine, propoxyphene, 6-monoacetyl morphine, morphine, fentanyl, tramadol, synthetic cannabinoids (e.g., K2, spice, etc.), and/or ketamine); and a single (1) sample validity testing strip that assays a sample for at least 3 (e.g., 3, 4, 5, 6, 7, 8, or more) sample characteristics including (e.g., 1) presence of an oxidant (e.g., bleach) in the sample; 2) presence of creatinine in the sample; 3) density (e.g., specific gravity) of the sample; and 4) hydrogen ion concentration (e.g., pH) of the sample). In some embodiments, a single sample validity testing strip assays a sample for 4 sample characteristics: (e.g., 1) presence of an oxidant (e.g., bleach) in the sample; 2) presence of creatinine in the sample; 3) density (e.g., specific gravity) of the sample; and 4) hydrogen ion concentration (e.g., pH) of the sample). This particular embodiment, comprising this particular combination of components and/or features, is illustrative and does not limit the technology. Accordingly, additional embodiments described herein comprise some or all or none of the features of this specific exemplary embodiment.

### Chamber

Embodiments of the assay device provided herein comprise a chamber 210 having an inner surface and an outer surface (FIG. 1A and FIG. 1B; FIG. 2A to FIG. 2G). In various embodiments, the chamber 210 comprises a symmetrical shape that is cylindrical, convex, conical, elliptical, square, or rectangular. Alternatively, in some embodiments, the chamber 210 comprises an asymmetrical shape, e.g., a peanut shape, kidney shape, or hybrid combinations thereof. Furthermore, embodiments of the chamber 210 have a size that is appropriate for the expected volumetric size of the sample to be collected and held within the chamber 210.

In some embodiments, the chamber 210 comprises an upper opening 211 defined by the upper portion of the chamber through which a sample can be introduced into the interior of the chamber 210. For example, embodiments provide that the chamber 210 comprises a side wall 212, a bottom wall 213, and a sample passage slot wall 214 (e.g., comprising a sample passage slot 230). In some embodiments, the sample passage slot wall 214 comprises a sample passage slot 230 and an umbrella feature 240 over the sample passage slot 230. In some embodiments, the sample passage slot wall 214 comprises a sample passage slot 230 and a hole 250 over the sample passage slot 230. In some embodiments, the sample passage slot wall 214 comprises a sample passage slot 230, an umbrella feature 240 over the sample passage slot, and a hole 250 over the sample passage slot 230. The umbrella feature 240 is provided to avoid specimen pouring directly into the reservoir 220 and/or to prevent flooding of the reservoir 220 with sample. The umbrella feature 240 protrudes from the sample passage slot wall 214 (e.g., to provide a “lip”) at least partially preventing the passage of sample through the sample passage slot 230 from above (e.g., when sample is being provided into the chamber). For example, the umbrella feature 240 protects the test strips from being stricken directly by sample during sample collection.

In some embodiments, the chamber side wall 212 and the specimen passage slot wall 214 are perpendicular, substantially perpendicular, and/or essentially perpendicular to the chamber bottom wall 213. In some embodiments, the chamber side wall 212 and the specimen passage slot wall 214 are tapered outwardly from the top to the bottom of the side wall 212 and the specimen passage slot wall 214. In some embodiments, the chamber side wall 212 and the specimen passage slot wall 214 are tapered inwardly from the top to the bottom of the side wall 212 and the specimen passage slot wall 214. In some embodiments, the taper ranges from approximately 1 to approximately 50 degrees (e.g., approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 degrees) from perpendicular. In some embodiments, the taper angle for the chamber side wall is from approximately 1 to 45 degrees (e.g., approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 degrees) from perpendicular. Alternatively, in some embodiments, the chamber side wall angle is tapered from approximately 1 to approximately 35 degrees (e.g., approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 degrees) from perpendicular. In some embodiments, the chamber side wall is tapered approximately 30 degrees from perpendicular.

In some embodiments, the chamber bottom wall 213 is substantially level. In some embodiments, the chamber bottom wall 213 is angled thereby directing flow of a sample towards the sample passage slot 230. The chamber 210 optionally includes a chamber orifice communicatively connecting the inside and the outside of the chamber 210. In some embodiments, the chamber 210 comprises a chamber orifice in the specimen passage slot wall 214 that is a hole 250.

In some embodiments, the chamber 210 comprises a chamber seal 300 to close the chamber upper opening 211. A non-exclusive list of mechanisms and methods that can be used to form seals useful in the present technology include, e.g., thermal welding, ultrasonic welding, vacuum sealing, compressive gaskets, screw-top lids, snap-top lids, compressive ring gaskets, gluing, compressive latch mechanisms, compressive spring mechanisms, snap couplings, bayonet couplings, zipping, hook and loop fasteners, screws, nails, bolting mechanisms, elastic band or bands, string and twine, wire, sliding mechanisms, plug or plugs, compressive clips, twist lids, epoxying, and tamper resistant mechanisms.

In some embodiments, the chamber 210 comprises a chamber indication structure 216 as described below.

In some embodiments, the chamber 210 comprises a tamper resistant seal or tamper evident seal to prevent tampering with the chamber opening 211 or to facilitate the detection of tampering with the chamber opening 211, respectively. In some embodiments, tamper evident seals prevent tampering with the chamber 210 prior to or after inserting a sample into the chamber 210 and, in some embodiments, prevent tampering with the reservoir seal 500. Tamper resistant seals can be of various types including a strap seal comprising a series of ratchet teeth arranged along the strap, with one end of the strap being secured to the outer surface of the chamber and the other end of the strap being lockably inserted into a lid hingeably attached to the upper end of the chamber to move the ratchet teeth sequentially past a resiliently deformable catch in the lid (see, e.g., U.S. Pat. No. 6,174,006, incorporated herein by reference). Other suitable tamper resistant and tamper evident seals are, e.g., foil seals, tape seals, locks, glue, epoxy, and/or hot wax seals as are well known in the art. Another tamper resistant or tamper evident seal that finds use in embodiments of the technology is a plastic heat shrunk band disposed around a sealed lid or a plastic heat-shrunk membrane disposed over a closure or opening. Attempted removal of or tampering with the closure causes the band to separate from the closure skirt, thus providing an indication of the tampering. Another method of providing a tamper resistant seal or tamper evident seal is to seal the present assay device comprising the closed chamber securely inside another container. Yet another tamper resistant seal comprises a series of sloped projections that irreversibly and unidirectionally engage with a series of sloped projections arrayed around the outer wall of the upper outer portion of the chamber. Preferably, a lid comprises a series of sloped projections that irreversibly and unidirectionally engage with a series of sloped projections arranged around the inner wall of the upper interior portion of the chamber.

In some embodiments, the technology comprises a chamber seal 300 that is a screw-lid (FIG. 3A to FIG. 3G). In embodiments comprising a screw-lid, a screw-lid comprises an array of external threads 320 that are unitary with and in proximity to the open top portion of the chamber side wall 212. The screw-lid threads 320 are configured to rotatably mate with similar threads 215 on chamber and thus hermetically seal the chamber 210 from outside contamination or adulteration and prevent, eliminate, and/or minimize the leakage or discharge of the contents of the chamber 210 during normal use conditions. In some embodiments, the chamber seal 300 optionally comprises an O-ring 330 that prevents unwanted leakage. In some embodiments, a torque of 15 to 30 pound-inch (e.g., 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9, or 30.0 pound-inch) is applied to the chamber seal (e.g., screw-lid) to seal the chamber 210. In some embodiments, a torque of less than or equal to 21.24 pound-inch (e.g., 240 Newton-centimeters) is applied to the chamber seal (e.g., screw-lid) to seal the chamber 210. In some embodiments, a torque of less than or equal to approximately 15 to 21.24 pound-inch (e.g., less than or equal to 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.2, 21.2, or 21.24 pound-inch) is applied to the chamber seal (e.g., screw-lid) to seal the chamber 210. In some embodiments, a torque of less than or equal to approximately 170 to 240 Newton-centimeters (e.g., less than or equal to approximately 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, or 240 Newton-centimeters) is applied to the chamber seal (e.g., screw-lid) to seal the chamber 210.

In some embodiments, the present technology comprises a chamber seal 300 (e.g., a screw-lid) comprising an indication structure 310 configured to produce a vibration (e.g., an audible sound and/or a haptic feedback) when the screw-lid securely seals the chamber 210, e.g., as described in U.S. Pat. No. 9,730,646, which is incorporated herein by reference in its entirety. In some embodiments, the present technology comprises a chamber seal 300 (e.g., a screw-lid) comprising an indication structure 310 configured to produce a vibration (e.g., an audible sound and/or a haptic feedback) when the screw-lid securely seals the chamber 210, e.g., as described in U.S. Pat. No. 9,730,646, which is incorporated herein by reference in its entirety. In particular, in some embodiments the chamber seal indication structure 310 is arranged on the screw-lid and a chamber wall indication structure 216 is arranged on a chamber wall. In use, when the screw-lid rotates to close the chamber opening, the two indication structure components 310 and 216 deform each other by pressing and then suddenly detaching from each other, producing the vibration when the components move back to their original state (e.g., the state prior to deformation). Preferably the two indication structure components 310 and 216 neither contact each other, nor other objects or parts, while vibrating. In a specific embodiment, the indication structure components 310 and 216 are arranged on the assay device 100 to create an indication sound that is a “pop”, “bang”, “boom”, “ding-ding”, “crackle”, or any other proper sound.

In some embodiments, a torque of 15 to 30 pound-inch (e.g., 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9, or 30.0 pound-inch) deforms one or both of the indication structure components 310 and/or 216 to produce the vibration (e.g., indication sound or haptic feedback). In some embodiments, a torque of less than or equal to 21.24 pound-inch (e.g., 240 Newton-centimeters) deforms one or both of the indication structure components 310 and/or 216 to produce the vibration (e.g., indication sound or haptic feedback). In some embodiments, a torque of less than or equal to approximately 15 to 21.24 pound-inch (e.g., less than or equal to 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.2, 21.2, or 21.24 pound-inch) deforms one or both of the indication structure components 310 and/or 216 to produce the vibration (e.g., indication sound or haptic feedback). In some embodiments, a torque of less than or equal to approximately 170 to 240 Newton-centimeters (e.g., less than or equal to approximately 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, or 240 Newton-centimeters) deforms one or both of the indication structure components 310 and/or 216 to produce the vibration (e.g., indication sound or haptic feedback).

In some embodiments, the vibration produces an indication sound that has an intensity of equal to or greater than 60-85 dB (e.g., equal to or greater than 60.0, 60.5, 61.0, 61.5, 62.0, 62.5, 63.0, 63.5, 64.0, 64.5, 65.0, 65.5, 66.0, 66.5, 67.0, 67.5, 68.0, 68.5, 69.0, 69.5, 70.0, 70.5, 71.0, 71.5, 72.0, 72.5, 73.0, 73.5, 74.0, 74.5, 75.0, 75.5, 76.0, 76.5, 77.0, 77.5, 78.0, 78.5, 79.0, 79.5, 80.0, 80.5, 81.0, 81.5, 82.0, 82.5, 83.0, 83.5, 84.0, 84.5, or 85.0 dB), 65-85 dB (e.g., equal to or greater than 65.0, 65.5, 66.0, 66.5, 67.0, 67.5, 68.0, 68.5, 69.0, 69.5, 70.0, 70.5, 71.0, 71.5, 72.0, 72.5, 73.0, 73.5, 74.0, 74.5, 75.0, 75.5, 76.0, 76.5, 77.0, 77.5, 78.0, 78.5, 79.0, 79.5, 80.0, 80.5, 81.0, 81.5, 82.0, 82.5, 83.0, 83.5, 84.0, 84.5, or 85.0 dB), 70-80 dB (e.g., equal to or greater than 70.0, 70.1, 70.2, 70.3, 70.4, 70.5, 70.6, 70.7, 70.8, 70.9, 71.0, 71.1, 71.2, 71.3, 71.4, 71.5, 71.6, 71.7, 71.8, 71.9, 72.0, 72.1, 72.2, 72.3, 72.4, 72.5, 72.6, 72.7, 72.8, 72.9, 73.0, 73.1, 73.2, 73.3, 73.4, 73.5, 73.6, 73.7, 73.8, 73.9, 74.0, 74.1, 74.2, 74.3, 74.4, 74.5, 74.6, 74.7, 74.8, 74.9, 75.0, 75.1, 75.2, 75.3, 75.4, 75.5, 75.6, 75.7, 75.8, 75.9, 76.0, 76.1, 76.2, 76.3, 76.4, 76.5, 76.6, 76.7, 76.8, 76.9, 77.0, 77.1, 77.2, 77.3, 77.4, 77.5, 77.6, 77.7, 77.8, 77.9, 78.0, 78.1, 78.2, 78.3, 78.4, 78.5, 78.6, 78.7, 78.8, 78.9, 79.0, 79.1, 79.2, 79.3, 79.4, 79.5, 79.6, 79.7, 79.8, 79.9, or 80.0 dB), and/or 75-80 dB (e.g., equal to or greater than 75.0, 75.1, 75.2, 75.3, 75.4, 75.5, 75.6, 75.7, 75.8, 75.9, 76.0, 76.1, 76.2, 76.3, 76.4, 76.5, 76.6, 76.7, 76.8, 76.9, 77.0, 77.1, 77.2, 77.3, 77.4, 77.5, 77.6, 77.7, 77.8, 77.9, 78.0, 78.1, 78.2, 78.3, 78.4, 78.5, 78.6, 78.7, 78.8, 78.9, 79.0, 79.1, 79.2, 79.3, 79.4, 79.5, 79.6, 79.7, 79.8, 79.9, or 80.0 dB).

In some embodiments, the vibration produces an indication sound that has an intensity of 60-90 dB (e.g., 60.0, 60.5, 61.0, 61.5, 62.0, 62.5, 63.0, 63.5, 64.0, 64.5, 65.0, 65.5, 66.0, 66.5, 67.0, 67.5, 68.0, 68.5, 69.0, 69.5, 70.0, 70.5, 71.0, 71.5, 72.0, 72.5, 73.0, 73.5, 74.0, 74.5, 75.0, 75.5, 76.0, 76.5, 77.0, 77.5, 78.0, 78.5, 79.0, 79.5, 80.0, 80.5, 81.0, 81.5, 82.0, 82.5, 83.0, 83.5, 84.0, 84.5, 85.0, 85.5, 86.0, 86.5, 87.0, 87.5, 88.0, 88.5, 89.0, 89.5, or 90.0 dB). In some embodiments, the vibration produces an indication sound that has an intensity of at least 10, 15, and/or 20 dB above background noise (e.g., at least 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0 dB above background noise).

In another embodiment of the assay device, the chamber seal comprises a snap-lid. Conventional plastic snap-lid closures comprise two basic elements—a main joint around which the lid pivots in relation to the lower part and one or several intermediate elements creating the snap effect. Such intermediate elements can be in the form of straps, triangles, or angled flexible springs or even longitudinally deformable tension spring elements. Snap-lid closures that are incorporated into embodiments of the present technology are described in, e.g., U.S. Pat. Nos. 3,688,942; 4,165,018; 4,177,930; 4,421,244; 4,476,993; 4,718,571; 4,966,302; 5,271,517; 5,294,015; 5,381,918; 228,031; 424,982; 3,837,518; 4,024,976; 4,111,329; 4,190,175; 4,493,432; 4,512,493; 4,646,926; 4,700,860; 4,711,364; 4,718,571; 4,807,771; 4,886,184; 5,002,198; 5,092,478; 5,111,947; 5,115,934; 5,207,340; and 5,271,517, each of which is herein incorporated by reference in its entirety. In some embodiments, the present technology comprises a chamber seal (e.g., a snap-lid) comprising an indication structure configured to produce a vibration (e.g., an audible sound and/or a haptic feedback) when the snap-lid securely seals the chamber, e.g., as described in U.S. Pat. No. 9,730,646, which is incorporated herein by reference in its entirety.

Embodiments provide that the chamber is manufactured using traditional manufacturing techniques known in the mechanical and manufacturing arts and provide that the chamber is constructed from various materials. These materials can include metal, silicon, glass, ceramic, plastic, and synthetic and natural polymers or any combination thereof. In some embodiments of the technology, the chamber is manufactured from a polypropylene composite using an appropriate manufacturing method.

In some embodiments, the chamber is made from a polymer. In some embodiments, the chamber comprises components made of a thermoplastic, a specialty plastic, a thermoset, and/or an engineering plastic. Thermoplastics include but are not limited to polyamideimide (PAD, polyethersulfone (PES), polyarylsulfone (PAS), polyetherimide (PEI), polyarylate (PAR), polysulfone (PSO), polyamide (PA), polycarbonate (PC), styrene-maleic anhydride (SMA), chlorinated PVC (CPVC), poly(methylmethacrylate) (PMMA), styrene-acrylonitrile (SAN), polystyrene (PS), acrylonitrile-butadiene-styrene (ABS), poly(ethyleneterephthalate) (PET), poly(vinylchloride) (PVC), polyetherketone (PEK), polyetheretherketone (PEEK), polytetrafluoroethylene (PTFE), poly(phenylene sulfide) (PPS), liquid crystal polymer (CCP), nylon-6,6, nylon-6, nylon-6,12, nylon-11, nylon 12, acetal resin, low and high density polypropylene (PP), high density polyethylene (HDPE), low density polyethylene (LDPE), polystyrene, ethylene-vinyl acetate, poly-vinyl-acetate, polyacrylic, etc., or a copolymer or a combination thereof. Specialty plastics include but are not limited to fluorocarbon polymers and infusible film products such as Kapton, Upilex polyimide film etc., a copolymer or a combination thereof. Thermosets include but not are limited to phenolics, epoxies, urea-formaldehyde, silicones, etc., a copolymer or a combination thereof. Engineering plastics include but are not limited to acetyl resins, polyamide, polyetherimides, polyesters, liquid crystal polymers, polycarbonate resins, poly(phenylene ether) alloys, polysulfone resins, polyamideimide resins, etc., a copolymer or a combination thereof. In some embodiments the chamber comprises high impact polystyrene. In some embodiments, the chamber is constructed from polystyrene (e.g., high impact polystyrene) using similar methods known in the art of plastics construction.

Methods of manufacturing can include but are not limited to milling, casting, blowing, spinning, injection molding, machining, and three-dimensional printing. In some embodiments of the present invention, the chamber is substantially transparent so that a user can observe a sample in the chamber interior by observation of the outside surface of the chamber. In some embodiments, the chamber is constructed separately from the reservoir. In some embodiments, the chamber and reservoir are constructed from a single mold. When constructed separately, the chamber may be joined with the reservoir by the manufacturer or at the point of care or prior to use. Assembly may be performed by aligning the chamber with the reservoir and may involve permanently affixing the chamber to the reservoir using glue, interlocking surfaces (e.g., snaps), and the like. Alternatively, the chamber may be reversibly affixed to the reservoir.

The size of the chamber is appropriate to meet or exceed the expected volumetric size of the sample to be contained and/or held within the chamber. As a lower limit, embodiments provide that the chamber volume is sufficiently large to transfer an adequate volume of a sample to the test device, considering adhesive forces between the materials of construction and the sample that reduce transfer of the sample to the reservoir. As an upper limit, embodiments provide that the chamber volume is sufficiently small to prevent the sample from overloading the reservoir (and, consequently, the test device and/or test strips) due to forces exerted from a sample entering the reservoir when the chamber is full. In exemplary embodiments, the chamber comprises a size to accommodate sample volumes greater than approximately 1.0 milliliter, 0.1 milliliter, 0.01 milliliter, 0.001 milliliter, or approximately 0.0001 milliliter and is manufactured to accommodate volumes less than approximately 1 milliliter, 5 milliliters, 10 milliliters, 50 milliliters, 100 milliliters, 250 milliliters, 500 milliliters, 750 milliliters, 1,000 milliliters, or approximately 2,000 milliliters. The volume to accommodate a gaseous sample may require a smaller or greater volume than the volume required for a liquid sample.

### Reservoir

Embodiments of the assay device 100 provided herein comprise a reservoir 220 having an inner surface and an outer surface (FIG. 1A and FIG. 1B; FIG. 2A to FIG. 2G). In various embodiments, the reservoir 220 is configured to accept a portion of the sample delivered from the chamber 210 through the specimen passage slot 230 and to allow the testing of said portions of the sample for the presence, absence, concentration, and/or amount of the analyte. Embodiments provide that the reservoir 220 is sufficiently large to accommodate a test device (e.g., comprising test device panel 400 and a plurality of test strips). Embodiments provide that the test device (e.g., comprising the test device panel 400) is positioned within the reservoir 220 and the reservoir 220 has an appropriate size to trap a volume of gas (e.g., air) that limits the portion of the sample entering the reservoir 220 to a desirable level that prevents the test device or test strip from being overloaded with a sample. One of ordinary skill in the art may test various shapes and sizes of the reservoir to assess their applicability by observing the height of a sample within the sealed reservoir. In some embodiments, the test device (e.g., comprising the test device panel 400 and a plurality of test strips) is inserted in the reservoir 220 during assembly by the manufacturer. In some embodiments, the test device is inserted in the reservoir 220 at the point of care. To facilitate assembly, in some embodiments the top part of the reservoir 220 comprises an opening 221 (e.g., a slot or slit) to permit the functional engagement of a test strip or test device with the reservoir 220 and thus provide the test device 100 as described herein. Alternatively, in some embodiments, the test device is inserted between two halves of a reservoir 220 and fused closed.

In some embodiments, the opening 221 of the reservoir 220 is hermetically sealed or sealed air tight with a reservoir seal 500. In some embodiments, a suitable reservoir seal comprises a plug, film (e.g., a metal film), and/or a self-adhesive seal made of paper, wax paper, plastic materials, thin metal films, metallicized plastic, or paper. In some embodiments, the reservoir seal 500 comprises laminated layers of, e.g., paper, metal foil, plastic, etc. In some embodiments, the reservoir seal 500 optionally further comprises a scored cover made integral with the adjacent reservoir material such that the scoring allows the scored cover area to be removed through breakage of the scored areas. In some embodiments, the reservoir seal 500 is not removable from the reservoir 220 after insertion of the test device into the reservoir 220 and placement of the reservoir seal 500. Thus, in some embodiments, the test device is permanently sealed inside the reservoir 220 by the reservoir seal 500.

Embodiments provide that the reservoir is manufactured using traditional manufacturing techniques known in the mechanical and manufacturing arts and provide that the reservoir is constructed from various materials. These materials can include metal, silicon, glass, ceramic, plastic, and synthetic and natural polymers or any combination thereof. In some embodiments, the reservoir is made from a polymer. In some embodiments, the reservoir comprises a thermoplastic, a specialty plastic, a thermoset, and/or an engineering plastic. Thermoplastics include but are not limited to polyamideimide (PAD, polyethersulfone (PES), polyarylsulfone (PAS), polyetherimide (PEI), polyarylate (PAR), polysulfone (PSO), polyamide (PA), polycarbonate (PC), styrene-maleic anhydride (SMA), chlorinated PVC (CPVC), poly(methylmethacrylate) (PMMA), styrene-acrylonitrile (SAN), polystyrene (PS), acrylonitrile-butadiene-styrene (ABS), poly(ethyleneterephthalate) (PET), poly(vinylchloride) (PVC), polyetherketone (PEK), polyetheretherketone (PEEK), polytetrafluoroethylene (PTFE), poly(phenylene sulfide) (PPS), liquid crystal polymer (CCP), nylon-6,6, nylon-6, nylon-6,12, nylon-11, nylon 12, acetal resin, low and high density polypropylene (PP), high density polyethylene (HDPE), low density polyethylene (LDPE), polystyrene, ethylene-vinyl acetate, poly-vinyl-acetate, polyacrylic, etc., or a copolymer or a combination thereof. Specialty plastics include but are not limited to fluorocarbon polymers and infusible film products such as Kapton, Upilex polyimide film etc., a copolymer or a combination thereof. Thermosets include but not are limited to phenolics, epoxies, urea-formaldehyde, silicones, etc., a copolymer or a combination thereof. Engineering plastics include but are not limited to acetyl resins, polyamide, polyetherimides, polyesters, liquid crystal polymers, polycarbonate resins, poly(phenylene ether) alloys, polysulfone resins, polyamideimide resins, etc., a copolymer or a combination thereof. In some embodiments, the reservoir comprises high impact polystyrene. In some embodiments of the technology, the reservoir is manufactured from a polypropylene composite using an appropriate manufacturing method. In some embodiments, the reservoir is constructed from polystyrene (e.g., high impact polystyrene) using similar methods known in the art of plastics construction.

Methods of manufacturing can include but are not limited to milling, casting, blowing, spinning, injection molding, machining, and three-dimensional printing. In some embodiments of the present invention, the reservoir is substantially transparent so that a test device present within the reservoir may be visualized by observation of the outside surface of the reservoir. In some embodiments, the reservoir is constructed separately from the chamber. In some embodiments, the reservoir and chamber are constructed from a single mold. When constructed separately, the reservoir may be joined with the chamber by the manufacturer or at the point of care or prior to use. Assembly may be performed by aligning the reservoir with the chamber and may involve permanently affixing the reservoir to the chamber using glue, interlocking surfaces (e.g., snaps), and the like. Alternatively, the reservoir may be reversibly affixed to the chamber.

In some embodiments of the present technology, the reservoir 220 accepts and engages a test device panel 400 to provide one or more test strips in a testably functional arrangement, e.g., the assay device 100 is configured to contact the appropriate sampling region of the test strips with a portion of the sample at the bottom of the test device 100. In some embodiments, the test device is permanently sealed inside the reservoir 220 by the reservoir seal 500.

### Sample Passage Slot and Reservoir Seal

Embodiments of the technology provide an assay device 100 comprising a chamber 210 and a reservoir 220 fluidly connected by a sample passage slot 230 (see, e.g., FIG. 2G to FIG. 2K). Accordingly, the sample passage slot 230 functions to provide the chamber 210 and the reservoir 220 to be in direct fluid communication and to allow a portion of a sample collected in the chamber 210 to flow into the reservoir 220 and contact the test device within the reservoir 220. The sample passage slot 230 is generally in the form of an aperture positioned at the bottom of the chamber. In some embodiments, the sample passage slot 230 is not rectangular. For instance, in some embodiments, the technology provides an assay device comprising a trapezoidal, triangular, or arched sample passage slot.

In some embodiments (e.g., as shown in FIG. 2H), the trapezoidal sample passage slot 230 comprises a first (e.g., bottom) base 231, a second (e.g., top) base 232, a first leg 233, a second leg 234, a first bottom angle 235, a second bottom angle 236, a first top angle 237, and a second top angle 238. In some embodiments, the trapezoidal passage slot 230 comprises a first vertical segment 239A and a second vertical segment 239B (e.g., wherein the first vertical segment 239A and the second vertical segment 239B clip portions of the trapezoid at the first bottom angle 235 and the second bottom angle 236).

In some embodiments, the lengths of the two parallel or substantially parallel sides (“bases”; see, e.g., FIG. 2H, bases 231 and 232) of the trapezoidal sample passage slot are in a ratio of from 1:4 to 80:100. That is, a first base 231 of the trapezoidal sample passage slot 230 (e.g., that is parallel or substantially parallel to a second base 232 of said trapezoidal sample passage slot 230) has a length that is from approximately 25% to approximately 80% (e.g., approximately 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80%) of the length of the second base 232 of the trapezoidal sample passage slot 230.

In some embodiments, the trapezoidal sample passage slot 230 has the shape of an acute trapezoid (e.g., the trapezoidal sample passage slot comprises two adjacent acute angles on its longer base edge) (see, e.g., FIG. 2H). In some embodiments, the trapezoidal sample passage slot has the shape of an isosceles trapezoid (e.g., a trapezoid where the legs 233 and 234 have the same length, the bottom angles 235 and 236 have the same measure, and the top angles 237 and 238 have the same measure).

In some embodiments, the trapezoidal sample passage slot is a shape that comprises at least one acute angle (e.g., FIG. 2H, the bottom angles 235 and 236). In some embodiments, the trapezoidal sample passage slot is a shape that comprises at least one obtuse angle (e.g., FIG. 2H, the top angles 237 and 238). In some embodiments, the trapezoidal sample passage slot is a shape that comprises two acute angles (e.g., near the bottom wall of the body of the device; e.g., FIG. 2H, bottom angles 235 and 236) and two obtuse angles (e.g., further from the bottom wall of the body of the device than the two acute angles; e.g., FIG. 2H, top angles 237 and 238). In some embodiments, the trapezoidal sample passage slot comprises a shape having an angle (e.g., a bottom angle 235 and/or 236) of from 1 to 89 degrees (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, or 89 degrees). In some embodiments, the trapezoidal sample passage slot comprises a shape having an angle (e.g., a top angle 237 and/or 238) of from 91 to 179 degrees (e.g., 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, or 179 degrees). In some embodiments, the trapezoidal sample passage slot comprises a shape having two acute angles (e.g., bottom angles 235 and 236) near the bottom wall of the body of the device (e.g., two angles of 1 to 89 degrees (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, or 89 degrees)) and two obtuse angles (e.g., top angles 237 and 238) further from the bottom wall of the body of the device than the two acute angles (e.g., two angles of from 91 to 179 degrees (e.g., 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, or 179 degrees).

In some embodiments, the trapezoidal sample passage slot 230 has a height (e.g., the perpendicular distance between the bases 231 and 232) that is from 2 to 15 mm (e.g., 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50, 9.75, 10.00, 10.25, 10.50, 10.75, 11.00, 11.25, 11.50, 11.75, 12.00, 12.25, 12.50, 12.75, 13.00, 13.25, 13.50, 13.75, 14.00, 14.25, 14.50, 14.75, or 15.00 mm). See, e.g., dimension c of FIG. 2G.

In some embodiments, a first (“bottom”) base 231 (FIG. 2H) of the trapezoidal sample passage slot (e.g., the base of the trapezoidal sample passage slot that is near the bottom wall of the body of the device) has a length of from 20 to 40 mm (e.g., 20.00, 20.25, 20.50, 20.75, 21.00, 21.25, 21.50, 21.75, 22.00, 22.25, 22.50, 22.75, 23.00, 23.25, 23.50, 23.75, 24.00, 24.25, 24.50, 24.75, 25.00, 25.25, 25.50, 25.75, 26.00, 26.25, 26.50, 26.75, 27.00, 27.25, 27.50, 27.75, 28.00, 28.25, 28.50, 28.75, 29.00, 29.25, 29.50, 29.75, 30.00, 30.25, 30.50, 30.75, 31.00, 31.25, 31.50, 31.75, 32.00, 32.25, 32.50, 32.75, 33.00, 33.25, 33.50, 33.75, 34.00, 34.25, 34.50, 34.75, 35.00, 35.25, 35.50, 35.75, 36.00, 36.25, 36.50, 36.75, 37.00, 37.25, 37.50, 37.75, 38.00, 38.25, 38.50, 38.75, 39.00, 39.25, 39.50, 39.75, or 40.00 mm). See, e.g., dimension b of FIG. 2G.

In some embodiments, a second (“top”) base 232 (FIG. 2H) of the trapezoidal sample passage slot (e.g., the base of the trapezoidal sample passage slot that is further from the bottom wall of the body of the device than the bottom base) has a length of from 5 to 25 mm (e.g., 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50, 9.75, 10.00, 10.25, 10.50, 10.75, 11.00, 11.25, 11.50, 11.75, 12.00, 12.25, 12.50, 12.75, 13.00, 13.25, 13.50, 13.75, 14.00, 14.25, 14.50, 14.75, 15.00, 15.25, 15.50, 15.75, 16.00, 16.25, 16.50, 16.75, 17.00, 17.25, 17.50, 17.75, 18.00, 18.25, 18.50, 18.75, 19.00, 19.25, 19.50, 19.75, 20.00, 20.25, 20.50, 20.75, 21.00, 21.25, 21.50, 21.75, 22.00, 22.25, 22.50, 22.75, 23.00, 23.25, 23.50, 23.75, 24.00, 24.25, 24.50, 24.75, or 25.00 mm). See, e.g., dimension a of FIG. 2G.

In some embodiments, the sample passage slot is arched, e.g., comprising the shape of a half-circle or other portion of a circle or a portion of an ellipse (e.g., in some embodiments the sample passage slot has a shape known in the geometry arts as a “segment” of a circle or a “segment” of an ellipse).

In some embodiments, the sample passage slot is triangular (see, e.g., FIG. 2I and FIG. 2J). In some embodiments (e.g., as shown in FIG. 2I and FIG. 2J), the triangular sample passage slot comprises a base and two arms. In some embodiments, the triangular passage slot 230 comprises a first vertical segment and a second vertical segment (e.g., wherein the first vertical segment and the second vertical segment clip portions of the triangle at the bottom angles). In some embodiments, the trapezoidal sample passage slot 230 has the shape of an isosceles triangle (e.g., a triangle comprising two sides (e.g., the two arms) that have the same length and two angles that are the same). In some embodiments, the triangular sample passage slot comprises at least one acute angle (e.g., FIG. 2I, the bottom angles). In some embodiments, the trapezoidal sample passage slot is a shape that comprises at least one obtuse angle (e.g., FIG. 2I, the top angle). In some embodiments, the triangular sample passage slot is a shape that comprises two acute angles (e.g., near the bottom wall of the body of the device; e.g., FIG. 2I, bottom angles) and one obtuse angle (e.g., the top angle further from the bottom wall of the body of the device than the two acute angles; e.g., FIG. 2I). In some embodiments, the triangular sample passage slot comprises a shape having an angle of from 1 to 89 degrees (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, or 89 degrees). In some embodiments, the triangular sample passage slot comprises a shape having an angle of from 91 to 179 degrees (e.g., 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, or 179 degrees).

In some embodiments, the triangular sample passage slot 230 has a height (e.g., the perpendicular distance from the base to the top) that is from 2 to 15 mm (e.g., 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50, 9.75, 10.00, 10.25, 10.50, 10.75, 11.00, 11.25, 11.50, 11.75, 12.00, 12.25, 12.50, 12.75, 13.00, 13.25, 13.50, 13.75, 14.00, 14.25, 14.50, 14.75, or 15.00 mm). See, e.g., FIG. 2I.

In some embodiments, the base of the triangular sample passage slot (e.g., near the bottom wall of the body of the device) has a length of from 20 to 40 mm (e.g., 20.00, 20.25, 20.50, 20.75, 21.00, 21.25, 21.50, 21.75, 22.00, 22.25, 22.50, 22.75, 23.00, 23.25, 23.50, 23.75, 24.00, 24.25, 24.50, 24.75, 25.00, 25.25, 25.50, 25.75, 26.00, 26.25, 26.50, 26.75, 27.00, 27.25, 27.50, 27.75, 28.00, 28.25, 28.50, 28.75, 29.00, 29.25, 29.50, 29.75, 30.00, 30.25, 30.50, 30.75, 31.00, 31.25, 31.50, 31.75, 32.00, 32.25, 32.50, 32.75, 33.00, 33.25, 33.50, 33.75, 34.00, 34.25, 34.50, 34.75, 35.00, 35.25, 35.50, 35.75, 36.00, 36.25, 36.50, 36.75, 37.00, 37.25, 37.50, 37.75, 38.00, 38.25, 38.50, 38.75, 39.00, 39.25, 39.50, 39.75, or 40.00 mm). See, e.g., FIG. 2I.

In some embodiments that comprise a triangular sample passage slot (see, e.g., FIG. 2I), the body does not comprise a vent hole (e.g., the body is vent hole-free). In some embodiments, the top of the triangular sample passage slot functions as a vent hole as described herein for embodiments that comprise a vent hole that is separate from the sample passage slot.

In some embodiments, the sample passage slot is arched, e.g., comprising the shape of a half-circle or other portion of a circle or a portion of an ellipse (e.g., in some embodiments the sample passage slot has a shape known in the geometry arts as a “segment” of a circle or a “segment” of an ellipse).

In some previous assay devices, the sample passage slot had a small size that alone did not promote the flow of sample from the chamber to the reservoir. Accordingly, in some previous assay devices a membrane was provided on the device floor to promote the flow of sample from the chamber to the reservoir. To address the issue of sample flow, some assay devices were provided with a larger sample passage slot to allow more sample to flow to the reservoir. However, the larger sample passage slot also increased the possibility that the sample will flood the reservoir if the assay device is tilted. For some previous assay devices, the maximal angle at which the device could be tilted without flooding the test strips was 23°.

In contrast, embodiments of the present technology comprise a sample passage slot having a trapezoidal or triangular shape (see, e.g., FIG. 2G to FIG. 2K), which allows the assay device to be tilted to a higher angle of incline (e.g., up to approximately 30°; see, e.g., FIG. 2J and FIG. 2K) than previous devices without flooding the test strips. Further, the sample passage slot design and/or sample passage slot size as provided by the present technology increases and/or maximizes flow of sample into the reservoir without use of a membrane on the floor of the assay device to promote flow of sample from the chamber to the reservoir. As used herein, the test device is vertical when the major vertical axis through the top of the device to the bottom aligns (e.g., essentially and/or substantially aligns) with a vector indicating the force of gravity. For instance, in some embodiments, the test strips of the assay device are not flooded when the assay device is tilted from vertical (e.g., at an angle of up to approximately 30° (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30° from vertical). See FIG. 2J and FIG. 2K.

In some embodiments, the reservoir seal 500 and sample passage slot 230 (e.g., trapezoidal or arched sample passage slot) control access and flow of the sample or a portion of the sample to the reservoir 220. In particular, the reservoir seal 500 prevents excess sample from entering the reservoir 220 by trapping air or a gas within the reservoir 220. The trapped air creates a downward pressure against the sample and therefore limits the amount of sample passing through the specimen passage slot 230. A typical urine sample will rise up to about 22 mm (e.g., approximately 20 to 25 mm) when the assay is conducted at approximately 0.8 to 1.2 atm (e.g., 0.80, 0.85, 0.90, 0.95, 1.00, 1.05, 1.10, 1.15, or 1.20 atm).

### Chamber, Reservoir, and Specimen Passage Slot Configurations

Embodiments of the technology provide an assay device 100 comprising the chamber 210 and reservoir 220 in a number of different arrangements. For example, in some embodiments, the chamber 210 and reservoir 220 form a single unit (e.g., a “body” 200 comprises both a chamber 210 and a reservoir 220 integrated into a single unit). In some embodiments, the chamber and reservoir are separate units. In some embodiments, the reservoir is configured to be attachable with and/or is attached to the separate chamber. Attaching the reservoir to the chamber may require alignment of a chamber sample passage slot and a reservoir sample passage slot thereby providing a functional sample passage slot. Suitable means of attaching the chamber to the reservoir include, e.g., thermal welding, ultrasonic welding, vacuum sealing, compressing gaskets, screw mechanisms, snap couplings, gluing, compressive latching mechanisms, compressive spring mechanisms, bayonet couplings, zipping, hook and loop fasteners, screws, nails, bolting mechanisms, elastic band or bands, string and twine, wire, sliding mechanisms, compressive clips, and epoxying.

In some embodiments, the reservoir is configured to be removable from the chamber. Suitable means of ensuring removability include, e.g., the use of thermal welding, ultrasonic welding, vacuum sealing, compressing gaskets, screw mechanisms, snap couplings, gluing, compressive latching mechanisms, compressive spring mechanisms, bayonet couplings, zipping, hook and loop fasteners, screws, nails, bolting mechanisms, elastic band or bands, string and twine, wire, sliding mechanisms, compressive clips, and epoxying.

### Test Device

The test device engages the reservoir 220 and detects and/or measures the presence, absence, concentration, and/or amount of an analyte of interest. In some embodiments, the test device comprises a test device panel 400 comprising 6 or more channels 410 (e.g., 6, 7, 8, 9, 10, 11, 12, 13, 14, 15, or more channels) (FIG. 4A to FIG. 4H) and 6 or more test elements (e.g., 6, 7, 8, 9, 10, 11, 12, 13, 14, 15, or more test elements). In some embodiments, the test device panel 400 comprises 6 channels 410 configured to hold test elements. In some embodiments, the test device panel 400 comprises 7 channels 410 configured to hold test elements. In some embodiments, the test device panel 400 comprises 8 channels 410 configured to hold test elements. In some embodiments, the test device comprises 6 or more channels (e.g., 6, 7, 8, 9, 10, 11, 12, 13, 14, 15, or more channels). In some embodiments, the test device comprises 6 channels. In some embodiments, the test device comprises 7 channels. In some embodiments, the test device comprises 8 channels. In some embodiments, the test device comprises 9 channels. In some embodiments, the test device comprises 10 channels. In some embodiments, the test device comprises 11 channels. In some embodiments, the test device comprises 12 channels. In some embodiments, the test device comprises 13 channels. In some embodiments, the test device comprises 14 channels. In some embodiments, the test device comprises 15 channels. In some embodiments, the test device comprises more than 15 channels.

In some embodiments, the test device comprises 6 or more test elements (e.g., 6, 7, 8, 9, 10, 11, 12, 13, 14, 15, or more test elements). In some embodiments, the test device comprises 6 test elements. In some embodiments, the test device comprises 7 test elements. In some embodiments, the test device comprises 8 test elements. In some embodiments, the test device comprises 9 test elements. In some embodiments, the test device comprises 10 test elements. In some embodiments, the test device comprises 11 test elements. In some embodiments, the test device comprises 12 test elements. In some embodiments, the test device comprises 13 test elements. In some embodiments, the test device comprises 14 test elements. In some embodiments, the test device comprises 15 test elements. In some embodiments, the test device comprises more than 15 test elements.

In some embodiments, the dimension a shown in FIG. 4D is approximately 40 to 60 mm (e.g., 40, 41, 42, 43, 44, 45, 46, 47, 48, 49, 50, 51, 52, 53, 54, 55, 56, 57, 58, 59, or 60 mm (e.g., 50.00 mm)). In some embodiments, the dimension b shown in FIG. 4D is approximately 40 to 60 mm (e.g., 40, 41, 42, 43, 44, 45, 46, 47, 48, 49, 50, 51, 52, 53, 54, 55, 56, 57, 58, 59, or 60 mm (e.g., 47.00 mm)). In some embodiments, the dimension c shown in FIG. 4D is approximately 35 to 55 mm (e.g., 35, 36, 37, 38, 39, 40, 41, 42, 43, 44, 45, 46, 47, 48, 49, 50, 51, 52, 53, 54, or 55 mm (e.g., 43.20 mm)). In some embodiments, the dimension h shown in FIG. 4F is approximately 60 to 80 mm (e.g., 60, 61, 62, 63, 64, 65, 66, 67, 68, 69, 70, 71, 72, 73, 74, 75, 76, 77, 78, 79, or 80 mm (e.g., 69.50 mm)). In some embodiments, the dimension d shown in FIG. 4F is approximately 1 to 5 mm (e.g., 1.0, 1.5, 2.0, 2.5, 3.0, 3.5, 4.0, 4.5, or 5.0 mm (e.g., 3.00 mm)).

In some embodiments, the test device panel comprises a center element 420 as shown in FIG. 4H. In some embodiments of the test panel comprising a center element, the center element facilitates movement of the fluid specimen into the test strips.

In some embodiments, the test device panel 400 comprises a polymer. In some embodiments, the test device panel 400 comprises components made of a thermoplastic, a specialty plastic, a thermoset, and/or an engineering plastic. Thermoplastics include but are not limited to polyamideimide (PAI), polyethersulfone (PES), polyarylsulfone (PAS), polyetherimide (PEI), polyarylate (PAR), polysulfone (PSO), polyamide (PA), polycarbonate (PC), styrene-maleic anhydride (SMA), chlorinated PVC (CPVC), poly(methylmethacrylate) (PMMA), styrene-acrylonitrile (SAN), polystyrene (PS), acrylonitrile-butadiene-styrene (ABS), poly(ethyleneterephthalate) (PET), poly(vinylchloride) (PVC), polyetherketone (PEK), polyetheretherketone (PEEK), polytetrafluoroethylene (PTFE), poly(phenylene sulfide) (PPS), liquid crystal polymer (CCP), nylon-6,6, nylon-6, nylon-6,12, nylon-11, nylon 12, acetal resin, low and high density polypropylene (PP), high density polyethylene (HDPE), low density polyethylene (LDPE), polystyrene, ethylene-vinyl acetate, poly-vinyl-acetate, polyacrylic, etc., or a copolymer or a combination thereof. Specialty plastics include but are not limited to fluorocarbon polymers and infusible film products such as Kapton, Upilex polyimide film etc., a copolymer or a combination thereof. Thermosets include but not are limited to phenolics, epoxies, urea-formaldehyde, silicones, etc., a copolymer or a combination thereof. Engineering plastics include but are not limited to acetyl resins, polyamide, polyetherimides, polyesters, liquid crystal polymers, polycarbonate resins, poly(phenylene ether) alloys, polysulfone resins, polyamideimide resins, etc., a copolymer or a combination thereof. In some embodiments, the test device panel 400 comprises a polymer (e.g., polypropylene and/or polystyrene (e.g., high impact polystyrene)).

The test device of the present technology can comprise any test element known in the art. For example, in some embodiments, the test device comprises a test element that is a lateral flow detection device such as a “test strip”. In some embodiments, the test device comprises a microfluidic component (e.g., a paper-based microfluidic component (e.g., comprising cellulose and/or nitrocellulose), a polymer-based microfluidic component (e.g., comprising polydimethylsiloxane), etc.). Lateral flow detection devices (“test strips”) include but are not limited to: immunoassays, chemical assays, and enzymatic assays commonly known in the art (e.g., single antibody immunoassays, multiple antibody immunoassays, sandwich immunoassays, competitive immunoassays, non-competitive immunoassays, and the like). In some embodiments, the test device comprises a test element (e.g., a test strip) that provides an assay that utilizes a reagent such as, e.g., horseradish peroxidase, alkaline phosphatase, luciferase, and/or an antibody (e.g., an antibody conjugate, antibody fragment, fluorescently tagged antibody, modified antibody, labeled antibody (e.g., antibody labeled with colloidal gold, antibodies labeled with colored latex bead)), and the like, which are commonly known in the art. In some embodiments, a test device comprises a test element (e.g., a test strip) that involves a capture method whereby a mobile primary molecule (e.g., first antibody) binds the analyte and traverses the test element (e.g., test strip) until a second immobilized molecule (e.g., second antibody) captures the bound analyte. These binding interactions generally occur in a reagent zone and detection zone, respectively. Alternatively, in some embodiments an enzyme and enzyme substrate are positioned in opposing zones or the same zone. The present technology contemplates that a mobile reagent may be incorporated into a sample application zone to eliminate the need for a reagent zone along the test element (e.g., test strip). Results are generally provided (e.g., visually) in a detection zone. Although many devices utilize antibodies to capture analytes, any reaction that produces a detectable result on a test element (e.g., a test strip) is sufficient and can be incorporated into the present technology. For example, and as previously mentioned, enzymes bound to an analyte in the presence of a substrate may also provide a detectable result. Examples of some test strips that may be incorporated into embodiments of the present technology are found in the following U.S. Pat. Nos. 4,857,453; 5,073,484; 5,119,831; 5,185,127; 5,275,785; 5,416,000; 5,504,013; 5,602,040; 5,622,871; 5,654,162; 5,656,503; 5,686,315; 5,714,389; 5,766,961; 5,770,460; 5,916,815; 5,976,895; 6,248,598; 6,140,136; 6,187,269; 6,187,598; 6,228,660; 6,235,241; 6,306,642; 6,352,862; 6,372,515; 6,379,620; 6,403,383; 6,485,982; 6,565,808; 7,270,959; and 7,300,633, each of which is incorporated herein by reference. The one or more test strips can be of any shape and dimensions; e.g., in some embodiments the test strip is a rectangular test strip having a sample application zone positioned generally at the bottom of the test strip and a detection zone positioned above the pressurized sample level. Preferably a reagent zone is positioned above the point in which a sample rises within the reservoir, however this need not be the case. The one or more test strips can be used separately or can be arrayed on or in a common support such as a test card.

In some particular embodiments, the test device (e.g., test device panel of the test device) comprises 6 or more channels (e.g., 6, 7, 8, 9, 10, 11, 12, 13, 14, 15, or more channels) configured to hold 6 or more test strips (e.g., 6, 7, 8, 9, 10, 11, 12, 13, 14, 15, or more test strips). In some embodiments, the test device (e.g., test device panel of the test device) comprises 6 channels configured to hold test strips. In some embodiments, the test device (e.g., test device panel of the test device) comprises 7 channels configured to hold test strips. In some embodiments, the test device (e.g., test device panel of the test device) comprises 8 channels configured to hold test strips. In some embodiments, the test device (e.g., test device panel of the test device) comprises 9 channels configured to hold test strips. In some embodiments, the test device (e.g., test device panel of the test device) comprises 10 channels configured to hold test strips. In some embodiments, the test device (e.g., test device panel of the test device) comprises 11 channels configured to hold test strips. In some embodiments, the test device (e.g., test device panel of the test device) comprises 12 channels configured to hold test strips. In some embodiments, the test device (e.g., test device panel of the test device) comprises 13 channels configured to hold test strips. In some embodiments, the test device (e.g., test device panel of the test device) comprises 14 channels configured to hold test strips. In some embodiments, the test device (e.g., test device panel of the test device) comprises 15 channels configured to hold test strips. In some embodiments, the test device (e.g., test device panel of the test device) comprises more than 15 channels configured to hold test strips.

In some embodiments, the test device (e.g., test device panel of the test device) comprises 6 or more test strips (e.g., 6, 7, 8, 9, 10, 11, 12, 13, 14, 15, or more test strips). In some embodiments, the test device (e.g., test device panel of the test device) comprises 6 test strips. In some embodiments, the test device (e.g., test device panel of the test device) comprises 7 test strips. In some embodiments, the test device comprises 8 test strips. In some embodiments, the test device comprises 9 test strips. In some embodiments, the test device comprises 10 test strips. In some embodiments, the test device comprises 11 test strips. In some embodiments, the test device comprises 12 test strips. In some embodiments, the test device comprises 13 test strips. In some embodiments, the test device comprises 14 test strips. In some embodiments, the test device comprises 15 test strips. In some embodiments, the test device comprises more than 15 test strips.

The increased number of test strips provides a device configured to test for an increased number of analytes (e.g., drugs). In some embodiments, the present technology provides an assay device that simultaneously tests for the presence, absence, and/or quantity of 14 or more drugs of abuse (e.g., 14, 15, 16, 17, 18, 19, 20, 21, 22, 23, 24, 25, or more drugs of abuse) in a sample. In some embodiments, the technology provides a test device that indicates the presence, absence, concentration, and/or amount of one or more of amphetamine, barbiturate, benzodiazepine, buprenorphine, cocaine, tetrahydrocannabinol (THC), ethyl glucuronide, methadone, methamphetamine, 3,4-methylenedioxy-methamphetamine (MDMA), opiate, oxycodone, phencyclidine, propoxyphene, 6-monoacetyl morphine, morphine, fentanyl, tramadol, synthetic cannabinoids (e.g., K2, spice, etc.; see, e.g., Liu (2018) Am J Clin Pathol. 149(2): 105-116; Ford (2017) Trends Pharmacol Sci. 38(3): 257-276; Davidson (2017) Adv Pharmacol. 80: 135-168, each of which is incorporated herein by reference), and ketamine. In some embodiments, the technology provides a test device that indicates the presence, absence, concentration, and/or amount of 6-monoacetyl morphine or morphine and fentanyl. In some embodiments, the technology provides a test device that indicates the presence, absence, concentration, and/or amount of oxycodone, propoxyphene, and tramadol. In some embodiments, the technology provides a test device that indicates the presence, absence, concentration, and/or amount of any of the foregoing by measuring the presence, absence, concentration, and/or amount of a metabolite of any of the foregoing.

In some embodiments, the technology provides a test device that detects amphetamine present in a sample at a concentration of 500 ng/ml or more (e.g., 500, 550, 600, 650, 700, 750, 800, 850, 900, 950, or 1000 ng/ml or more). In some embodiments, the technology provides a test device that detects barbiturate present in a sample at a concentration of 300 ng/ml or more (e.g., 300, 350, 400, 450, 500, 550, 600, 650, 700, or 750 ng/ml or more). In some embodiments, the technology provides a test device that detects benzodiazepine present in a sample at a concentration of 300 ng/ml or more (e.g., 300, 350, 400, 450, 500, 550, 600, 650, 700, or 750 ng/ml or more). In some embodiments, the technology provides a test device that detects buprenorphine present in a sample at a concentration of 10 ng/ml or more (e.g., 10, 15, 20, 25, 30, 35, 40, 45, or 50 ng/ml or more). In some embodiments, the technology provides a test device that detects cocaine present in a sample at a concentration of 150 ng/ml or more (e.g., 150, 160, 170, 180, 190, 200, 210, 220, 230, 240, or 250 ng/ml or more). In some embodiments, the technology provides a test device that detects THC present in a sample at a concentration of 50 ng/ml or more (e.g., 50, 60, 70, 80, 90, 100, 110, 120, 130, 140, or 150 ng/ml or more). In some embodiments, the technology provides a test device that detects ethyl glucuronide present in a sample at a concentration of 500 ng/ml or more (e.g., 500, 550, 600, 650, 700, 750, 800, 850, 900, 950, or 1000 ng/ml or more). In some embodiments, the technology provides a test device that detects methadone present in a sample at a concentration of 300 ng/ml or more (e.g., 300, 350, 400, 450, 500, 550, 600, 650, 700, or 750 ng/ml or more). In some embodiments, the technology provides a test device that detects methamphetamine present in a sample at a concentration of 500 ng/ml or more (e.g., 500, 550, 600, 650, 700, 750, 800, 850, 900, 950, or 1000 ng/ml or more). In some embodiments, the technology provides a test device that detects 3,4-methylenedioxy-methamphetamine present in a sample at a concentration of 500 ng/ml or more (e.g., 500, 550, 600, 650, 700, 750, 800, 850, 900, 950, or 1000 ng/ml or more). In some embodiments, the technology provides a test device that detects opiates present in a sample at a concentration of 2000 ng/ml or more (e.g., 2000, 2050, 2100, 2200, 2250, 2300, 2350, 2400, 2450, or 2500 ng/ml or more). In some embodiments, the technology provides a test device that detects oxycodone present in a sample at a concentration of 100 ng/ml or more (e.g., 100, 110, 120, 130, 140, 150, 160, 170, 180, 190, or 200 ng/ml or more). In some embodiments, the technology provides a test device that detects phencyclidine present in a sample at a concentration of 25 ng/ml or more (e.g., 25, 30, 35, 40, 45, 50, 55, 60, 65, 70, or 75 ng/ml or more. In some embodiments, the technology provides a test device that detects propoxyphene present in a sample at a concentration of 300 ng/ml or more (e.g., 300, 350, 400, 450, 500, 550, 600, 650, 700, or 750 ng/ml or more). In some embodiments, the technology provides a test device that detects 6-monoacetyl morphine and/or morphine present in a sample at a concentration of 10 ng/ml or more (e.g., 10, 15, 20, 25, 30, 35, 40, 45, or 50 ng/ml or more). In some embodiments, the technology provides a test device that detects fentanyl present in a sample at a concentration of 20 ng/ml or more (e.g., 20, 25, 30, 35, 40, 45, 50, 55, 60, 65, 70, or 75 ng/ml or more). In some embodiments, the technology provides a test device that detects tramadol present in a sample at a concentration of 100 or 200 ng/ml or more (e.g., 100, 110, 120, 130, 140, 150, 160, 170, 180, 190, 200, 210, 220, 230, 240, 250, 260, 270, 280, 290, or 300 ng/ml or more). In some embodiments, the technology provides a test device that detects synthetic cannabinoids (e.g., K2, spice, etc.) present in a sample at a concentration of 30 ng/ml or more (e.g., 30, 35, 40, 45, 50, 55, 60, 65, 70, or 75 ng/ml or more). In some embodiments, the technology provides a test device that detects ketamine present in a sample at a concentration of 100 ng/ml or more (e.g., 100, 110, 120, 130, 140, 150, 160, 170, 180, 190, or 200 ng/ml or more). In some embodiments, the technology provides a test device that detects multiple drugs (e.g., 2, 3, 4, 5, or 6 drugs) on a single test strip. In some embodiments, the technology provides a test device and/or a test strip that detects any subset and/or any combination of any of the aforementioned drugs or drug metabolites. In some embodiments, the technology provides a test device that comprises a plurality of test strips (e.g., 6 or more test strips (e.g., 6, 7, 8, 9, 10, 11, 12, 13, 14, 15, or more test strips), wherein each test strip detects multiple drugs (e.g., each test strip detects 2, 3, 4, 5, or 6 drugs).

In some embodiments, the test strips comprise a sample pad that is larger than previous test strips. In some embodiments, the test strips comprise a sample pad that is more than 19 mm in length (e.g., approximately 25 mm in length (e.g., 20, 21, 22, 23, 24, 25, 26, 27, 28, 29, or 30 mm in length)). In some embodiments, the test strips are assembled from layers to decrease the susceptibility of the test strips to delamination. For example, in some embodiments, the overlap of a first layer (e.g., an adhesive tape) and a second layer (e.g., an immunochromatographic matrix on an impermeable backing layer) is increased to minimize and/or eliminate the delamination of the test strip. In some embodiments, the overlap of a first layer (e.g., an adhesive tape) and a second layer (e.g., an immunochromatographic matrix on an impermeable backing layer) is approximately 2 to 3 mm (e.g., 2.0, 2.1, 2.2, 2.3, 2.4, 2.5, 2.6, 2.7, 2.8, 2.9, or 3.0 mm). In some embodiments, the test strips are narrower than previous test strips, e.g., in some embodiments the test strips have a width of approximately 3.5 mm (e.g., 3.3, 3.4, 3.5, 3.6, or 3.7 mm). The narrower width of the test strips provides a test device comprising an increased number of test strips relative to previous test devices.

The test device further comprises a specimen validation testing strip configured to test for four aspects of sample validity simultaneously on said single strip, e.g., in some embodiments a test strip assays a sample for: 1) presence of an oxidant (e.g., bleach) in the sample; 2) presence of creatinine in the sample; 3) density (e.g., specific gravity) of the sample; and 4) hydrogen ion concentration (e.g., pH) of the sample.

### Methods of Manufacturing

In some embodiments, the technology relates to methods of manufacturing an assay device described herein. In some embodiments, the technology relates to methods of manufacturing one or more components (e.g., a chamber, reservoir, lid, and/or test device) of an assay device described herein. Methods of manufacturing can include but are not limited to milling, casting, blowing, spinning, injection molding, machining, and three-dimensional printing. In some embodiments, methods of manufacturing comprise steps performed manually. In some embodiments, methods of manufacturing comprise steps that are automated (e.g., performed by a machine or manually with machine assistance). In some embodiments, a chamber, a reservoir, a lid, and/or a test device is produced by automated injection molding. In some embodiments, an assay device is manually loaded on a transfer belt and/or assembly line for automated manipulation. In some embodiments, a label is affixed to the assay device by an automated label affixer component. In some embodiments, a test device panel is inserted into a reservoir of an assay device by an automated test device panel inserter component. In some embodiments, the reservoir seal is positioned on the reservoir by an automated reservoir seal placing component. In some embodiments, pressure and/or heat is applied to the reservoir seal to seal the reservoir by an automated reservoir seal sealing component. In some embodiments, a lid is placed on a chamber by an automated lid placing component and, in some embodiments, the lid is rotated by an automated lid rotating component to engage the lid securely to the chamber.

### Sample

In some embodiments, the assay device 100 described herein is configured to collect, hold, and/or assay a sample, including liquid samples as described herein. Alternatively, in some embodiments, the assay device 100 is configured to collect, hold, and/or assay other types of samples. For example, the sample may comprise a fine powdery material (e.g., talc, carbon black, and/or a pharmaceutical preparation) or a gas (e.g., argon or methane). In some embodiments, samples include atmospheric specimens that are tested for particulates or radioactive isotopes such as radon.

In some embodiments of the technology, the sample is a biological sample. Biological specimens include but are not limited to a sample from a subject such as an animal (e.g., a mammal (e.g., a primate (e.g., human))). A sample from a subject can be of any appropriate type, such as a sample of fluid, tissue, organ, or a combination thereof. The biological specimen can also be a sample of other biological material, such as food, including food such as material derived from plants or animals or combinations thereof. In some embodiments, the sample is processed prior to introduction into the chamber. In some embodiments, the chamber includes reagents for use in such processing. In some embodiments, a sample and reagent are combined within the chamber. In some embodiments, a reagent is used to process a sample, e.g., to digest a solid sample with appropriate reagents (e.g., chemicals, acids, bases, and/or enzymes (e.g., proteases)). In some embodiments, reagents are used to extract an analyte from a sample. For example, in some embodiments, the technology relates to extracting an antigen from a biological entity (e.g., an etiological agent (e.g., bacteria, parasites, viruses, or prions such as known in the art)).

While a number of different biological samples are suitable for collection and assay by the present technology, commonly collected samples are biological samples, including but not limited to fluid samples (e.g., urine, blood, serum, oral fluid (e.g., saliva), semen, secretions (e.g., vaginal secretions), central nervous system fluids (e.g., spinal fluid), lavages, and the like). However, the specimen can also be an environmental sample, such as a sample of soil, water, wastewater, landfill, or landfill leachate.

In some embodiments, the chamber 210 accommodates sample volumes of between approximately 0.0001 milliliter to approximately 1,000 milliliters. In some embodiments, the sample is diluted or concentrated depending on the concentration of the analyte and the sensitivity of the test device. As a general guideline the sample may be greater than 1.0 milliliter, 0.1 milliliter, 0.01 milliliter, 0.001 milliliter, or approximately 0.0001 milliliter and may be less than approximately 1 milliliter, 5 milliliters, 10 milliliters, 50 milliliters, 100 milliliters, 250 milliliters, 500 milliliters, 750 milliliters, 1,000 milliliters, or approximately 2,000 milliliters. However, the present technology envisions additional ranges depending on the needs of the user.

### Systems

In some embodiments, a system comprises an assay device 100 as described herein. For example, in some embodiments, the technology provides a system comprising an assay device 100 as described herein and an optical reader configured to record a result provided by the assay device on a detection zone. In some embodiments, the technology provides a system comprising an assay device 100 as described herein, an optical reader, and a computer configured to record, calculate, display, or communicate a result. In some embodiments, a system comprises a computer-based analysis program that translates the result (e.g., the presence, absence, concentration, and/or amount of one or more analytes) into an indicator for a user (e.g., a clinician, an employer, an insurance provider, a user). Embodiments provide methods for receiving, processing, and transmitting the result or indicator to and from laboratories conducting the assays, information providers, medical personal, and subjects. The result and/or indicator may be displayed to the clinician by any suitable method, e.g., a printed report or displayed to the clinician on a computer monitor.

### Uses

The technology finds use in a variety of applications settings. In some embodiments, the assay device described herein finds use in forensic applications. In some embodiments, the assay device finds use by an employer (e.g., to monitor drug use by employees onsite and/or offsite). In some embodiments, the assay device finds use in criminal justice (e.g., to monitor drug use by individuals on probation, parole, under house arrest, in post-incarceration rehabilitation, etc.) In some embodiments, the assay device finds use in insurance (e.g., to monitor insureds and/or to evaluate risk). In some embodiments, the assay device finds use in rehabilitation of drug users or addicts. In some embodiments, the technology finds use in a home, medical clinic, emergency room, or doctor's office.

Although the disclosure herein refers to certain illustrated embodiments, it is to be understood that these embodiments are presented by way of example and not by way of limitation.

### Example

A test device comprising a plurality of test strips configured to detect the presence of 1-14 or more drugs of abuse is inserted into the reservoir to provide an assay device. The reservoir seal is placed over the reservoir sealing the test device within the reservoir. A person in need of a drug-of-abuse test is given an embodiment of the present technology, referred to as “the urine cup” in this example. The person goes to the lavatory. In the lavatory, the person opens the top of the urine cup, urinates into the chamber of the urine cup, screws on the screw-lid of the urine cup until the indication structure indicates that the screw-lid has sealed the chamber (e.g., using a torque of less than or equal to 21.3 pound-inch), and gives the urine cup to a technician. The technician places the cup on a substantially horizontal surface such as a laboratory bench or a counter. When the person urinates into the chamber of the urine cup, a portion of the urine flows into the reservoir through the trapezoidal, triangular, or arched sample passage slot. The flow of urine pressurizes the air within the reservoir thereby limiting the flow of urine into the reservoir. After the urine flows into the reservoir, the urine contacts the sample application zones of the 6 or more test strips and single sample validity testing (SVT) strip and the sample migrates along the test strips and SVT strip. If present, one or more analytes pass through the reagent zones and bins to one or more labeled antibody. This binding complex continues to migrate to the detection zones where an immobilized antibody or specific binding molecule captures the analyte at a region different than the labeled antibody. The test results observable at the detection zone are viewed through the indicator windows of the test device within the sealed reservoir. The technician reports the results of the test and disposes of the used urine cup in the appropriate biohazard container.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. An assay device comprising:
a) a chamber configured for collecting and holding a sample, said chamber comprising an indication structure;
b) a reservoir comprising a test device; and
c) a non-rectangular sample passage slot fluidly connecting said chamber and said reservoir.

2. The assay device of claim 1 wherein said test device comprises 6 or more channels configured to hold 6 or more test strips.

3. The assay device of claim 1 wherein said non-rectangular sample passage slot has a trapezoidal or a triangular shape.

4. The assay device of claim 3 wherein said trapezoidal shape has a first base that is 20-40 mm long.

5. The assay device of claim 3 wherein said trapezoidal shape has a second base that is 5-25 mm long.

6. The assay device of claim 3 wherein said trapezoidal shape has a height of 2-15 mm.

7. The assay device of claim 3 wherein said trapezoidal shape is an isosceles trapezoid.

8. The assay device of claim 3 wherein said trapezoidal shape comprises two angles of 30 to 60 degrees.

9. The assay device of claim 1 wherein said non-rectangular sample passage slot has a shape that is an arch, segment of a circle, or segment of an ellipse.

10. The assay device of claim 1 further comprising a lid configured to seal said chamber.

11. The assay device of claim 10 wherein said indication structure is configured to produce a vibration when said lid is securely engaged with said chamber.

12. The assay device of claim 11 wherein a torque of at least 21.3 pound-inch is required to produce the vibration.

13. The assay device of claim 10 wherein said lid is a screw-lid.

14. The assay device of claim 1 wherein said test device further comprises a sample validity testing strip configured to test for at least three sample characteristics.

15. The assay device of claim 14 wherein said sample characteristics are pH, specific gravity, presence of creatinine, and presence of an oxidant.

16. The assay device of claim 1 wherein said assay device does not comprise a membrane on the bottom wall of said assay device.

17. The assay device of claim 1 wherein said chamber, said reservoir, and/or said test device comprises plastic.

18. (canceled)

19. The assay device of claim 1 wherein said test strips are configured to test for the presence, absence, amount, and/or concentration of drugs of abuse.

20. The assay device of claim 19 wherein drugs of abuse are selected from the set consisting of amphetamine, barbiturate, benzodiazepine, buprenorphine, cocaine, tetrahydrocannabinol, ethyl glucuronide, methadone, methamphetamine, 3,4-methylenedioxy-methamphetamine, opiate, oxycodone, phencyclidine, propoxyphene, 6-monoacetyl morphine, morphine, fentanyl, tramadol, synthetic cannabinoids, and ketamine.

21. The assay device of claim 1 wherein said test strips are configured to test for the presence, absence, amount, and/or concentration of drug or drug metabolite.

22. The assay device of claim 11 wherein said vibration provides an audio or haptic signal to a user of said assay device.

23. The assay device of claim 1 further comprising a sample.

24. The assay device of claim 1 further comprising a urine, oral fluid, blood, serum, or spinal fluid sample.

25. The assay device of claim 1 further comprising a reservoir seal providing an airtight seal for said reservoir.

26. The assay device of claim 25, wherein said reservoir seal, in cooperation with said non-rectangular specimen passage slot, controls fluid passage from said chamber to said reservoir.

27. The assay device of claim 25, wherein said reservoir seal, in cooperation with said non-rectangular specimen passage slot, prevents excess influx of a sample into said reservoir upon introduction of said sample into said chamber.

28-75. (canceled)
